(19) United States
(12) Patent Application Publication
(10) Pub. No.: US 2014/0206735 A1
(43) Pub. Date: Jul. 24, 2014

(54) HIGH DOSAGE TOPICAL METRONIDAZOLE AQUEOUS-BASED GEL FORMULATIONS AND THEIR USE TO TREAT ROSACEA

(71) Applicant: Medicis Pharmaceutical Corporation, Bridgewater, NJ (US)

(72) Inventors: Michael T. Nordsiek, Wayne, PA (US); Kodumudi S. Balaji, Lansdale, PA (US)

(73) Assignee: Medicis Pharmaceutical Corporation, Bridgewater, NJ (US)

(21) Appl. No.: 14/136,280

(22) Filed: Dec. 20, 2013

Related U.S. Application Data
(60) Provisional application No. 61/740,414, filed on Dec. 20, 2012.

(51) Int. Cl. A61K 31/4164

(52) U.S. Cl. CPC A61K 31/4164
USPC 514/398

(57) ABSTRACT

The present disclosure provides aqueous-based gel formulations of metronidazole useful for a variety of purposes, including topical application as a therapeutic approach towards the treatment of individuals suffering from and/or diagnosed with rosacea.

### 1. FIELD

The present disclosure relates to aqueous-based gel formulations of metronidazole and their uses, for example to treat rosacea.

### 2. BACKGROUND

Rosacea is a chronic skin disease that can affect the cheeks, nose, eyes, chin and forehead. It is characterized by recurrent episodes of flushing, erythema (redness), papules (pimples), pustules, and telangiectasias (permanent distended blood capillary vessels with a spidery appearance). See, e.g., Crawford et al., 2004, “Rosacea I. Etiology, Pathogensis and Subtype Classification,” J Am Acad Dermatology 51(2):327-241 (“¹⁴Crawford 2004”); Korting & Schöllmann, 2009, “¹⁴Current Topical and Systemic Approaches to Treatment of Rosacea,” J Eur Acad Dermatology & Venereology 23(8);876-882 (“Korting 2009”); Powell, 2005, “¹⁴Clinical Practice: Rosacea,” N Engi J Med 352(8):793-803 (“Powell 2005”). It can cause inflammation of the eyes or eyelids, or both, as well as thickening of the skin, specially of the nose, called rhinophyma. As with most chronic skin conditions, rosacea requires long-term treatment. Treatment options include orally administered antibiotics such as tetracyclines (e.g., doxycline and minocycline) or azithromycin ((see, e.g., Powell 2005; Korting 2009; Culp & Schienfeld, 2009, “Rosacea: A Review,” P&T 34(1):38-45 (“¹⁴Culp 2009”); Elewski et al., 2011, “Rosacea—Global Diversity and Optimized Outcome: Proposed International Consensus from the Rosacea International Expert Group,” J Eur Acad Dermatology & Venereology 25(2):188-200 (“Elewski 2011”)), topical application of agents such as metronidazole (“MTZ”), azelaic acid, clindamycin, permethrin, tretinoin, sulfacetamide with sulphur and benzoyl peroxide alone or in combination with erthromycin or clindamycin (Powell 2005; Culp 2009; Korting 2009; Elewski 2011), light-based therapies such as pulsed dye laser or intense pulsed light ((Kawana et al., 2007, “Objective Evaluation of the Effect of Intense Pulsed Light on Rosacea and Solar Lentigines by Spectrophotometric Analysis of Skin Color,” Dermatol Surgery 33(4):449-545 (“Kawana 2007”); Culp 2009; Korting 2009)), and combinations of these and other therapies.

In cases where a small number of papules and pustules are present, topical rather than systemic therapy is considered to be the first-line of treatment (see, e.g., van Zuuren et al., 2011, 2009726-0023 “Interventions for Rosacea (Review), The Cochrane Collaboration, published by John Wiley & Sons, Ltd. (“van Zuuren 2011”). One such topical treatment is metronidazole (MTZ).

MTZ is available from numerous sources as oral tablets and capsules, injectable solutions, 0.75 wt % and 1.0 wt % topical lotions, creams and gels, and 0.75 wt % vaginal gels. The topical creams and lotions and the 1.0 wt % gels are generally indicated for the treatment or rosacea, whereas the 0.75 wt % vaginal gels are indicated for the treatment of bacterial vaginosis.

Despite the number of treatment options available for managing rosacea, an estimated 16 million American suffer from this disease. Accordingly, new treatment options for managing rosacea would be desirable. In particular, it would be desirable and beneficial to have available new topical formulations that could be applied less frequently and/or provide improved management of symptoms, as compared to currently available topical therapies.

### 3. SUMMARY

It has been surprisingly discovered that formulating metronidazole (“MTZ”) in certain aqueous-based gel vehicles at concentrations higher than those currently employed to treat rosacea yields topical formulations having properties that in clinical use are expected to yield treatment regimens and/or outcomes that are superior to those achieved with currently available topical 0.75% and/or 1.0% by weight (wt %) MTZ gels. For example, as will be discussed in more detail below, it has been surprising discovered that “high dosage” topical MTZ aqueous-based gels comprising about 1% to about 2% by weight of MTZ, and in specific embodiments about 1.5% by weight MTZ, deliver significantly higher than expected local concentrations of MTZ, and significantly higher local concentrations of MTZ than are delivered by a 0.75 wt % MTZ gel in in vitro skin permeation experiments carried out with ¹⁴C-labeled MTZ and human cadaver skin. Moreover, significantly more MTZ is retained in the stratum corneum and other superficial layers of the skin, while at the same time less penetrates through the skin and is taken up in receiver fluid. While not intending to be bound by any theory of operation, it is believed that the MTZ retained in the stratum corneum may act as a “reservoir” or depot for prolonged or sustained release of MTZ over time, permitting fewer applications as a function of time as compared to currently available topical MTZ gels, and/or treatment regimens of shorter duration and/or improved clinical outcomes. Since less MTZ penetrates the skin, it is believed that the high dosage MTZ aqueous-based gels described herein may provide these clinical benefits with fewer adverse side effects.

Accordingly, in one aspect, the present disclosure provides novel “high dosage” MTZ aqueous-based gels that can be applied topically to subjects to treat, among other things, rosacea. The high dosage MTZ aqueous-based gels generally comprise about 1% to about 2% by weight MTZ, in some specific embodiments, about 1.0%, about 1.1%, about 1.2%, about 1.3%, about 1.4%, about 1.5%, about 1.6%, about 1.7%, about 1.8%, about 1.9%, or about 2.0% by weight MTZ, and one or more gelling polymers. The MTZ may be included in the gel in the form of a free base or as a salt, such as a salt formed with a pharmaceutically acceptable acid.

The gels typically include a total quantity of gelling polymer(s) sufficient to yield a viscosity in the range of about 20,000 mPa to about 500,000 mP, in some embodiments about 30,000 mP to about 400,000 mPa, in still other embodiments about 40,000 mPa to about 350,000 mPa, in yet other embodiments about 50,000 mPa to about 300,000 mPa, in still other embodiments about 75,000 mPa, to about 200,000 mPa, and in some specific embodiments about 75,000 mPa to about 125,000 mPa measured at 25° C. using the controlled shear ramp rate method, a Bohlin CVO 100 rheometer and the rheometer settings noted in Table 7. Depending upon the specific gelling polymer(s) used, aqueous-based gels having viscosities in this range are generally achieved by including in the gels a total quantity of gelling polymer(s) ranging from about 0.5% to about 5% by weight. In some specific embodiments, the high dosage MTZ aqueous-based gels described herein include about 1% to about 3% by weight total gelling polymer(s), and in some specific embodiments about 1%, about 1.5% or about 2% by weight total gelling polymer(s).

Skilled artisans will appreciate that different gelling polymers exhibit different degrees of viscosity under different conditions. The specific gelling polymer(s) selected are not critical for success and can be selected from amongst any polymers capable of forming gels in aqueous-based solutions. Exemplary suitable gelling polymers are described in more detail in the Detailed Description section. All of these gelling polymers can be used singly or in combinations.

Skilled artisans will also appreciate that for many topical applications, it may be desirable for the gel to exhibit a certain degree of “tackiness” or mucoadhesion so that the gel is not easily removed by incidental washing or perspiration. In these instances, it may be desirable to select gelling polymers that exhibit mucoadhesive properties, or to include in the gels agents, whether gelling or not, that exhibit mucoadhesive properties. Mucoadhesive gelling polymers are well-known, and include by way of example cross-linked polymers of acrylic acid (e.g., carbomers and polycarbophils) and various cellulosic polymers (e.g., hydroxyethyl cellulose, hydroxypropyl cellulose, and hydroxypropyl methylcellulose). Skilled artisans will be able to select gelling polymers having degrees of mucoadhesion suitable for particular applications.

In a specific exemplary embodiment of high dosage MTZ aqueous-based gels suitable for topical application for the treatment of rosacea, the one or more gelling polymer(s) are cross-linked polymers of acrylic acid and/or cellulosic polymers. In one specific exemplary embodiment, the gelling polymer(s) are selected from the group consisting of a hydroxyalkyl cellulose, a carbomer, a polycarbophil and mixtures thereof. In another specific exemplary embodiment, the gelling polymer(s) is a carbomer, such as, for example, carbomer 974P.

The high dosage MTZ aqueous-based gels also comprise a solvent system for the MTZ. It is well-known that MTZ presents solubility problems when attempting to formulate gels useful for topical administration with excipients approved by the FDA for use in humans. Others have attempted to solubilize MTZ in aqueous solutions at concentrations of greater than 0.75% or 1.0% by weight. For example, U.S. Pat. No. 6,881,726 describes the use of cyclodextrin and beta-cyclodextrin to enhance the solubility of MTZ in aqueous-based solutions. U.S. Pat. No. 7,348,317 describes the additional use of niacin and niacinamide to enhance the solubility of MTZ in aqueous-based solutions. Indeed, currently-available 1 wt % MTZ topical gels indicated for the treatment of rosacea list betadex (a beta cyclodextrin) and niacinamide as ingredients. Still others have attempted to increase the concentration of MTZ in aqueous-based solutions using surfactants. Any of these solvent systems can be used to solubilize the MTZ to the desired concentration in the high dosage MTZ aqueous-based gels described herein.

It has, however, been surprisingly discovered that MTZ can be solubilized in novel solvent systems at concentrations sufficient to yield high dosage MTZ aqueous-based gels comprising about 1% to about 2% by weight MTZ, and in some specific embodiments about 1.5% by weight MTZ, without having to use additional solubility enhancing compounds, such as those described above. This is advantageous, because agents such as cyclodextrins and beta-cyclodextrins in many instances enhance the solubility of compounds in aqueous solutions by forming complexes, resulting in complex formulations where the compound is not uniformly dissolved in the formulation. Moreover, agents such as niacin and niacinamide may themselves have undesired pharmacological properties at certain concentrations, and agents such as surfactants can cause gels to be multiphasic. Thus, in certain embodiments, the novel solvent systems described herein can be substantially free of any such solubility enhancing compounds.

Accordingly, also provided is a novel solvent system useful for preparing high dosage MTZ aqueous-based gels as described herein. The novel solvent system yields high dosage MTZ aqueous-based gels that are homogeneous, i.e., the MTZ is dissolved in the gel, and that are shelf-stable for long periods of time, such as six months or more, when stored at a temperature in the range of about 25° C. to about 40° C. Indeed, a specific exemplary embodiment of a high dosage MTZ aqueous-based gel comprising 1.3% by weight MTZ has been found to be stable for a period of at least 18 months when stored at temperatures of 25° C. and 40° C.

The novel solvent system generally utilizes one or more solvents that collectively have a saturated MTZ solubility at 25° C. that is sufficient to yield a gel containing the desired concentration of MTZ. Generally, such solvents will each have a saturated MTZ solubility of at least about 10 mg/g at 25° C., although skilled artisans will appreciate that when solvents are selected that have appreciably higher saturated MTZ solubilities, the novel solvent system may include solvents with lower saturated MTZ solubilities. A number of solvents having suitable saturated MTZ solubilities that may be used as components of the novel solvent system are provided in the Examples section.

Certain classes of solvents have been discovered to have saturated MTZ solubilities at 25° C. of at least about 20 mg/g. For example, it has been discovered that certain alcohols, for example, lower aliphatic alcohols such as ethanol and lower aromatic alcohols such as benzyl alcohol, certain diols, such as, for example lower aliphatic diols (including, for example, lower aliphatic glycols such as ethylene glycol and propylene glycol) and certain polyethers, such as, for example, polyoxyalkylenes having molecular weights in the range of about 200 to about 600 (“lower polyoxyalkylene”) (including, for example, PEG 400) have saturated MTZ solubilities at 25° C. in this range. It is expected that other solvents with similar physicochemical properties will have similar saturated MTZ solubilities at 25° C., for example, carbonates of lower aliphatic diols and alkyl ethers of isosorbides. Certain lower aromatic alcohols have significantly higher saturated MTZ solubilities at 25° C. For example, benzyl alcohol has a saturated MTZ solubility of about 72 mg/g at 25° C. It is also expected that other aromatic alcohols, for example, phenoxyethanol, may exhibit similarly high saturated MTZ solubiliations at 25° C. All of these solvents, alone and in various combinations, may be used in the novel solvent systems to solubilize MTZ in the high dosage gels described herein.

In some embodiments, the novel solvent system comprises at least one solvent having a saturated MTZ solubility at 25° C. of at least about 50 mg/g and optionally one or more solvents having a saturated MTZ solubility at 25° C. of at least about 20 mg/g. In some embodiments, the novel solvent system comprises at least one first solvent having a saturated MTZ solubility at 25° C. of at least about 50 mg/g and one or more second solvents that collectively have a saturated MTZ solubility at 25° C. of at least about 20 mg/g, in some embodiments in a range of about 20 mg/g to about 25 mg/g. In some specific embodiments, each of the second solvents has a saturated MTZ solubility at 25° C. of at least about 20 mg/g, in some embodiments in a range of about 20 mg/g to about 25 mg/g.

In some specific embodiments, the novel solvent system comprises from about 1 wt % to about 5 wt % of the at least one first solvent, with the remaining about 95 wt % to about 99 wt % being composed of the one or more second solvents. In some specific embodiments, the solvent system comprises about 1.0 wt %, about 1.5 wt %, about 2.0 wt %, about 2.5 wt %, about 3.0 wt %, about 3.5 wt %, about 4.0 wt %, about 4.5 wt % or about 5.0 wt % (or any range bracketed by any of these values) of the at least one first solvent and the remainder being composed of the one or more second solvents.

In some specific embodiments, the at least one first solvent is a lower aromatic alcohol and the one or more second solvent is a lower aliphatic polyol, a lower polyether and/or a lower polyoxyalkylene.

The one or more lower aromatic alcohol can be a phenolic, primary, secondary, or tertiary alcohol, and therefore may include aliphatic or heteroaliphatic groups in addition to an aromatic group. The one or more lower aliphatic polyols, lower polyethers, and lower polyoxyalkylenes can be saturated or unsaturated and can include primary, secondary and/or tertiary alcohol groups. Exemplary lower aromatic alcohols, lower aliphatic polyols, lower polyethers, and lower polyoxyalkylenes useful in this specific embodiment of the novel solvent system and high dosage MTZ aqueous-based gels described herein are described in more detail in the Detailed Description section.

The solvent system will generally represent about 30% to about 65% by weight of the high dosage MTZ aqueous-based gel, and in some specific embodiments about 55% to about 62% by weight. The exact amount used will depend, at least in part, on the desired concentration of MTZ to be included in the gel. In some other specific embodiments, the solvent system represents about 40% to about 65% by weight of the high dosage MTZ aqueous-based gel.

The quantities of the components of the solvent system can also be described with respect to the high dosage MTZ aqueous-based gels. In some embodiments, the high dosage MTZ aqueous-based gels comprise from about 1.3% to about 2% by weight total lower aromatic alcohol(s) (for example, benzyl alcohol), about 15% to about 25% by weight total lower aliphatic diol(s) (for example, propane-1,2-diol), and about 15% to about 40% by weight total polyoxyalkylene(s) (for example, PEG 400). In some specific embodiments, the high dosage MTZ aqueous-based gels comprise about 1.3% to about 1.5% by weight total lower aromatic alcohol(s) (for example, benzyl alcohol), about 20% by weight total lower aliphatic diol(s) (for example, propane-1,2-diol) and about 35% to about 40% by weight polyoxyalkylene(s) (for example, PEG-400). Other specific embodiments are described in the Detailed Description section, as are additional solvents and agents useful for solubilizing MTZ in connection with the high dosage MTZ aqueous-based gels described herein.

The pH of the high dosage MTZ aqueous-based gels described herein should generally match the pH of the intended area of application and/or fall within a range that does not cause excessive irritation at the site of application. Accordingly, for gels intended for topical application to areas of skin affected by rosacea, the pH should generally be in the range of about pH 4 to about pH 8. In some embodiments the high dosage MTZ aqueous-based gels have a pH in the range of about pH 5.2 to about pH 5.5. The pH can be adjusted and/or maintained with the aid of acids, bases and buffers, as is well-known in the art. For example, the pH of the gel may be adjusted and/or maintained by utilizing as the aqueous phase of the gel a buffer having a suitable normality and pH. Alternatively, the pH of the aqueous phase may be adjusted with an acid or base prior to adding the gelling agent. After gelling, the pH may be adjusted further with an acid or base. This latter method may be advantageous for preparing gels utilizing gelling agents that gel most efficiently outside the desired pH range of the resultant gel. For example, carbomers gel most efficiently around neutral pH. High dosage MTZ aqueous-based gels suitable for topical application to skin utilizing carbomer gelling agents may be prepared by adjusting the pH of the gelling solution to approximately neutral for gelling and then adjusting the pH of the resultant gel to within a range of about pH 5.2 to about pH 5.5 with an acid.

The high dosage MTZ aqueous-based gels described herein can also include other additional components, such as, for example, one or more preservatives, as is well-known in the art. When used, preservative(s) should generally represents no more than about 1% or 2% by weight of the high dosage MTZ aqueous-based gel. The choice of preservative(s) is not critical. Suitable useful preservatives are described in more detail in the Detailed Description section. In some specific embodiments the one or more preservative(s) are esters of 4-hydroxy benzoic acid, also known as parabens. Suitable parabens include lower alkyl esters of 4-hydroxy benzoic acid, such as, for example, methyl 4-hydroxybenzoate (methyl parben), ethyl 4-hydroxybenzoate (ethyl paraben) and propyl 4-hydroxy-benzoate (propyl paraben).

Skilled artisans will appreciate that solvents used to solubilize the MTZ in the high dosage MTZ aqueous-based gels described herein may also have preservative properties. For example, benzyl alcohol has well known preservative properties. When used as a solvent in the novel solvent system, the preservative properties can be used to advantage. Indeed, gels including solvents with preservative properties need not necessarily include additional preservatives. Gels that utilize the preservative properties of solvent system solvents should, in cases where the solvent may degrade and/or oxidize over time, include an amount of overage that takes into account the degradation and/or oxidation such that the gel retains an amount of undegraded and/or unoxidized solvent having effective preservative properties after a desired period of time. For example, benzyl alcohol is known to oxidize to benzaldehyde, which does not have preservative properties. In embodiments of the high dosage MTZ gels described herein that employ benzyl alcohol as an MTZ solvent and as a preservative, an amount of benzyl alcohol should be included in the gel that in addition to solubilizing the MTZ, yields a preservative effect for the duration of the expected shelf life of the gel. Overage amounts of benzyl alcohol, or other solvents employed in the solvent system as preservatives, can be determined based upon the degradation properties and kinetics of the particular solvent under the desired conditions of storage.

Embodiments of high dosage MTZ aqueous-based gels that include solvents having preservative properties may also include one or more additional preservatives, and/or preservatives designed to protect the solvent from degradation and/or oxidation. For example, in the case of benzyl alcohol, the high dosage MTZ gels described herein may include one or more additional preservatives that have antioxidant properties, in part to protect the benzyl alcohol from oxidation. In a specific embodiment, high dosage MTZ gels comprising benzyl alcohol or other solvents that oxidize include one or more parabens as additional preservatives. In some specific embodiments the high dosage MTZ aqueous-based gels comprise about 0.1% by weight total parabens, and in some specific embodiments about 0.02% by weight methyl paraben and about 0.08% by weight propyl paraben.

As is evident from its name, individuals suffering from rosacea oftentimes have red or rosy-colored skin in the affected area. Indeed, a common symptom of rosacea is erythema. To help conceal or camouflage the redness, high dosage gels designed for use in treating rosacea may also include a dye or pigment, typically in the green area of the visible spectrum (e.g., reflecting light of about 510 nm).

The high dosage MTZ gels described herein also include water, either in the form of pure water or in the form of an aqueous buffer. Typically, the amount of water included in the gel will be less than about 70%, more typically less than about 60% or 50% by weight, and in some specific embodiments, in the range of about 35% to about 40% by weight.

As noted above, several embodiments of high dosage MTZ aqueous-based gels prepared with the novel solvent system described herein deliver unexpectedly high local concentrations of MTZ, and significantly higher local concentrations of MTZ than a commercially available 0.75% MTZ gel in in vitro skin permeation experiments carried out in a Franz Cell with ¹⁴C-radiolabeled MTZ and human cadaver skin (and also silicone membranes). Indeed, as is described in more detail in the Examples section, virtually every high dosage MTZ gel tested in this experiment locally delivered in the range of about 25 to about 55-fold more MTZ, when normalized for concentration, than the 0.75 wt % MTZ gel. Moreover, despite the higher local MTZ concentrations delivered by the high dosage MTZ aqueous-based gels described herein, significantly less MTZ was observed in receiver fluid as compared to the 0.75 wt % MTZ gel in in vitro skin permeation experiments carried out in a Franz Cell with ¹⁴C-labeled MTZ and human cadaver skin. As is described in more detail in the Detailed Description section, virtually every high dosage MTZ gel tested in this experiment delivered in the range of about 1 to about 20-fold less MTZ, when normalized for concentration, than the 0.75 wt % MTZ gel. Moreover, significantly more MTZ was absorbed into the stratum corneum with the high dosage MTZ aqueous-based gels described herein in this experiment. For example, with reference to FIGS. 1-8, it can be seen that in this Franz cell experiments carried out with ¹⁴C-labeled MTZ and human cadaver skin, about 25-fold to about 55-fold more MTZ was retained in the stratum corneum from the high dosage aqueous-based gels described herein as compared to a conventional 0.75% MTZ gel. Although not intending to be bound by any theory of operation, it is believed that these properties may yield significant benefits when the gels are used to topically treat skin diseases, such as rosacea. Specifically, it is believed that the high concentration of MTZ in the stratum corneum may act as a reservoir or depot of MTZ, permitting less frequent application than current gels, overall treatment regimens of shorter duration and/or improved clinical outcomes, while at the same time reducing systemic exposure, thereby reducing untoward side effects.

Accordingly, in some embodiments, the various components of the high dosage MTZ aqueous-based gels are selected so as to yield a high dosage gel in which the local levels of MTZ measured in an in vitro Franz cell skin permeation study carried out with ¹⁴C-labeled MTZ and human cadaver skin or a silicone membrane, when normalized for concentration, are at least about 25-fold higher, and in some specific embodiments about 55-fold higher, than the level measured with a conventional 0.75% MTZ gel, such as the 0.75% MTZ gel sold by Galderma under the trade name METROGEL® or the gel sold by Medicis under the trade name METROGEL VAGINAL®. In some embodiments, the various components of the high dosage MTZ aqueous-based gels are selected so as to yield a high dosage MTZ gel in which the level of MTZ measured in the stratum corneum in an in vitro Franz cell skin permeation study carried out with ¹⁴C-labeled MTZ and human cadaver skin, when normalized for concentration, are about 25-fold to about 55-fold higher than the level measured with 0.75% METROGEL®. In some embodiments, the various components of the high dosage MTZ aqueous-based gels are selected so as to yield a high dosage gel in which the levels of MTZ measured in an in vitro Franz cell skin permeation study carried out with human cadaver skin, when normalized for concentration, are at least about 1-fold lower, and in some specific embodiments about 20-fold lower, than the level measured with 0.75% METROGEL®. A specific in vitro Franz cell experiment that can be used for the comparative studies described in Example 4.

The high dosage MTZ aqueous-based gels described herein can be used for any purpose where treatment with MTZ is desirable. Due, at least in part, to the unexpected skin penetration properties discussed above, it is believed that the high-dosage gels described herein will be particularly useful for the topical treatment of skin disorders, such as rosacea.

Accordingly, in another aspect, the present disclosure provides methods of treating subjects suffering from and/or diagnosed with rosacea using the high dosage MTZ aqueous-based gels described herein. The methods generally involve applying topically to the affected area of the subject an amount of a high dosage MTZ aqueous-based gel as described herein sufficient to provide therapeutic benefit. The clinical criteria for diagnosis, as well as criteria for establishing therapeutic benefit, are described in more detail in the Detailed Description section. In some specific embodiments, the amount of high dosage MTZ gel applied in a single application suitable for covering about 64 cm² of area (e.g., the area of an average adult cheek) contains from about 3 mg to about 10 mg MTZ. In some specific embodiments, the amount of high dosage MTZ gel applied in a single application suitable for covering about 64 cm² of area contains from about 3.125 mg to about 5 mg MTZ, from about 3.5 mg to about 4 mg MTZ, and in some specific embodiments about 3.75 mg MTZ.

The frequency and duration of application can vary, and may depend upon the desired outcome. Generally, the gel may be applied once or twice per day for a period of about 4 to about 12 weeks, although since rosacea is a chronic disease, the high dosage gel can be applied once or twice per day indefinitely. While not intending to be bound by any particular theory, it is believed that the reservoir of MTZ delivered to the stratum corneum by the high dosage MTZ gels described herein may permit less frequent application than currently available gels and/or a shorter overall treatment regimen, while at the same time delivering the same degree of efficacy, or even improved efficacy.

Accordingly, it is expected that in some instances, the high dosage MTZ gels described herein may provide efficacy when applied once or twice a day for a period of only about 4 to about 10 weeks. In other embodiments it is expected that application less frequently than daily, for example, once every other day, or once every third day, may provide therapeutic benefit.

Rosacea may be characterized by periods of outbreak interspersed by periods where few or relatively few symptoms are experienced (“asymptomatic periods” or periods of remission). The asymptomatic periods may last for several days, several weeks, months, and even several months, in the latter case especially if common outbreak “triggers” are avoided. It is believed that the reservoir and/or depot properties of the high dosage MTZ aqueous-based gels described herein may also lengthen the asymptomatic periods experienced by subjects suffering from and/or diagnosed with rosacea. Accordingly, in some embodiments, a treatment regimen involves topically applying a high dosage MTZ aqueous-based gel once or twice per day, or less frequently, for a period of 4 to 12 weeks as described above, discontinuing therapy for a period of days, weeks, one month, two months, or even three months or longer, and then resuming therapy. Therapy may be advantageously resumed, in advance of exposure to common triggers of rosacea outbreak to possibly delay or ameliorate the severity of the outbreak.

Regardless of the frequency or overall duration of treatment, the area should preferably be washed prior to applying the gel, with any cosmetics or other skin products being applied after the MTZ gel.

For use, the high dosage MTZ aqueous-based gels can be packaged in any form that is convenient for the desired mode of application. In some specific embodiments, the high dosage MTZ aqueous-based gels may be packaged in unit dosage form, for example in tubes or other packages that contain an amount of gel suitable for a single application.

It should be understood that the above summary is not intended to describe every embodiment or every implementation of the various inventions disclosed herein. The Detailed Description and Examples section further exemplify illustrative embodiments. The various embodiments described herein are intended to be disclosed in combinations, as if each specific combination were explicitly disclosed. The Examples are representative only and should not be interpreted as exclusive, or limiting the scope of the various inventions disclosed herein.

### 4. BRIEF DESCRIPTION OF THE FIGURES

FIG. 1 provides a graph illustrating the amount ¹⁴C-labeled MTZ recovered from various layers of skin from exemplary high dosage MTZ gels containing 1.3 wt % MTZ and a 0.75 wt % METROGEL® control in an in vitro Franz cell experiment carried out with human cadaver skin (data are mean±SEM).

FIG. 2 provides a graph illustrating the data of FIG. 1 represented as a percentage of the total quantity of ¹⁴C-labeled MTZ applied to the skin.

FIG. 3 provides a graph of the data of FIG. 1 grouped by test gel.

FIG. 4 provides a graph of the data of FIG. 2 grouped by test gel.

FIG. 5 provides a graph illustrating the amount of ¹⁴C-labeled MTZ recovered from various layers of skin from exemplary high dosage MTZ gels containing 1.5 wt % MTZ and a 0.75% METROGEL® control in an in vitro Franz cell experiment carried out with human cadaver skin (data are mean±SEM; n=12).

FIG. 6 provides a graph illustrating the recovery data of FIG. 5 represented as a percentage of the total quantity of ¹⁴C-labeled MTZ applied to the skin.

FIG. 7 provides a graph illustrating certain recovery data of FIG. 5 plotted at a different scale.

FIG. 8 provides a graph illustrating certain recovery data of FIG. 6 plotted at a different scale.

FIG. 9 is a graph illustrating the MTZ recovered from various skin layers of exemplary high dosage MTZ aqueous-based gels and two control 0.75 wt % MTZ gels. Data are mean (±SD, n=5-6).

FIG. 10 is a graph illustrating the MTZ recovery data of FIG. 9, grouping together the MTZ recovered from the skin surface and stratum corneum.

FIG. 11 is a graph illustrating the amount of MTZ recovered from receiver fluid from the experiment of Example 5.

FIG. 12 is a graph illustrating the data of FIG. 11 plotted as a percentage of the total MTZ applied.

FIG. 13 is a graph illustrating the data of FIG. 11, following on the first 24 hours of the experiment.

### 5. DETAILED DESCRIPTION

A more complete appreciation of the various inventions disclosed herein, and many of the attendant advantages thereof, is provided by the detailed description that follows.

### 5.1. Definitions

As used herein throughout the specification and in the appended claims, the following terms and expressions are intended to have the following meanings:

The indefinite articles “a” and “an” and the definite article “the” are intended to include both the singular and the plural, unless the context in which they are used clearly indicates otherwise.

“At least one” and “one or more” are used interchangeably to mean that the article may include one or more than one of the listed elements.

Unless otherwise indicated, it is to be understood that all numbers expressing quantities, ratios, and numerical properties of ingredients, reaction conditions, and so forth, used in the specification and claims are contemplated to be able to be modified in all instances by the term “about”.

All parts, percentages, ratios, etc., herein are by weight unless indicated otherwise.

### 5.2. Detailed Description of Specific Exemplary Embodiments

As noted in the Summary, the present disclosure provides, among other things, high dosage metronidazole (“MTZ”) aqueous-based gels that are useful for topical delivery of MTZ to treat various indications, and in a specific embodiment for topical application as a therapeutic approach towards the treatment of subjects suffering from, or diagnosed with, rosacea. The high dosage MTZ aqueous-based gels generally comprise MTZ and one or more gelling polymers.

MTZ, also known as 2-(2-methyl-5-nitro-1H-imidazol-1-yl)ethanol, is a well-known antimicrobial agent, having activity against anaerobic Gram-negative bacilli including Fusobacterium species and Bacteroides species (e.g., B. fragilis, B. distasonis, B. ovatus, B. thetaioaomicron, and B. vulgates); anaerobic Gram-positive bacilli including Clostridium species and susceptible strains of Eubacterium; and anaerobic Gram-positive cocci including Peptostreptococcus species. It also has anti-inflammatory and antioxidant properties. The exact nature by which MTZ provides benefit in the context of rosacea is not presently known, but certain authors suggest that MTZ exerts its therapeutic efficacy via its anti-inflammatory and antioxidant properties. See, e.g., Del Rosso, 2007, “The Role of Skin Care and Maintaining Proper Barrier Function in the Management of Rosacea,” Cosmetic Dermatology 20(8):485-490 (“Del Rocco 2007”); Narayanan et al., 2007, “Scavenging Properties of Metronidazole on Free Oxygen Radicals in a Skin Lipid Model System,” J Pharmacy Pharmacol. 59(8):1125-1130 (“Narayanan 2007”); Elewski 2011.

MTZ can be included in the high dosage gels described herein in the form of a free base or as a salt formed with pharmaceutically acceptable acids. Inorganic acids suitable for forming pharmaceutically acceptable salts include, by way of example and not limitation, hydrohalide acids (e.g., hydrochloric acid, hydrobromic acid, hydriodic, etc.), sulfuric acid, nitric acid, phosphoric acid and the like. Organic acids suitable for forming pharmaceutically acceptable salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-tuluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

MTZ is available commercially or can be synthesized by well-known methods.

The high dosage MTZ aqueous-based gels described herein generally comprise MTZ in amounts ranging from about 1% by weight to about 2% by weight. In specific embodiments, the gels described herein comprise about 1.0%, about 1.1%, about 1.2%, about 1.3%, about 1.4%, about 1.5%, about 1.6%, about 1.7%, about 1.8%, about 1.9% or about 2.0% by weight MTZ.

The gels also comprise one or more gelling polymers that impart the gels with their gel-like properties. A variety of polymers that form gels in aqueous-based solutions that are suitable for use in the aqueous-based gels described herein are known in the art, and include by way of example and not limitation, polysaccharide hydrocolloids (including, for example, mucilages, gums such as xanthan gum and tragacanth, and glucans), celluloses and modified celluloses (including, for example, alkyl celluloses, hydroxyalkyl celluloses, carboxy celluloses and sodium carboxy celluloses), poloxomers (copolymers of polyoxyethylene and polyoxypropylene, also known as PLURONICS®), carbomers (cross-linked polymers of acrylic acid), polycarbophils (polymers of polyacrylic acid cross-linked with divinyl glycol), veegum (magnesium aluminum silicate), polyvinyl alcohol (PVA), gelatin, sodium alginate and polyvinylpyrrolidone (PVP). Exemplary suitable mucilages can be found, for example, in Malviya et al., 2011, “Applications of Mucilages in Drug Delivery—A Review,” Advan Biol Res 5(1):1-7, and the references cited therein, the disclosures of which are incorporated herein by reference.

In some specific embodiments, the gelling polymer(s) are cross-linked polymers of acrylic acids, such as for example carbomers or polycarbophils, and/or cellulosic polymers. Suitable cellulosic polymers include, but are not limited to, carboxy methyl cellulose (CMC), methylcellulose, ethyl cellulose, hydroxylethyl cellulose (HEC or HHX), hydroxylpropyl cellulose (HPC) and hydroxylpropyl methyl cellulose (HPMC). Suitable carbomers include, but are not limited to the various polymers sold under the trade name CARBOPOL® by Lubrizol Advanced Materials, Cleveland, Ohio, including, for example, CARBOPOL® homopolymers (polymers of acrylic acid cross-linked with allyl sucrose or allyl pentaerythritol) such as CARBOPOL® 71G NF, CARBOPOL® 971P NF, CARBOPOL® 974P NF, CARBOPOL® 980 NF, and CARBOPOL® 981 NF; CARBOPOL® copolymers (polymers of acrylic acid and C10-C30 alkyl acrylate cross-linked with allyl pentaerythritol) such as PEMULEN™ TR-1 NF and PEMULEN™ TR-2 NF; CARBOPOL® interpolymers (carbomer homopolymers or copolymers that contain a block copolymer of polyethylene glycol and long chain alkyl acid ester) such as CARBOPOL® ETD 2020 NF and CARBOPOL® Ultrez 10 NF; “traditional” carbomers such as carbomer 934 (CARBOPOL® 934 NF), carbomer 934P (CARBOPOL® 934P NF), carbomer 940 (CARBOPOL® 940 NF), carbomer 941 (CARBOPOL® 941 NF) and carbomer 1342 (CARBOPOL® 1342P NF); and polycarbophil (NOVEON® AA-1 USP).

Any of these and/or other gelling polymers can be used, singly or in combinations, in the high dosage MTZ aqueous-based gels described herein.

The one or more gelling polymers are typically used in quantities such that the resultant high dosage MTZ aqueous-based gel has a viscosity in the range of about 60,000 mPa to about 500,000 mPa (measured at 25° C.), using the controlled shear rate ramp method of Example 2, a Bohlin CVO 100 rheometer and the rheometer settings of Table 7. In some specific embodiments, quantities of gelling polymers are used that yield gels having viscosities in the range of about 75,000 mPa to about 125,000 mPa, although gels having higher viscosities, for example in the range of about 200,000 mPa to about 400,000 mPa, or about 300,000 mP are expected to be useful. Specific methods for measuring the viscosity and of the gels are provided in Example 2.

Depending upon the gelling polymer(s) selected, high dosage MTZ aqueous-based gels having viscosities in the above ranges will typically comprise about 0.5% to about 5% by weight total gelling polymer(s). Specific exemplary embodiments comprise about 1% to about 3% by weight, and in some cases about 1%, about 1.5% or about 2% by weight, total gelling polymer. Exemplary high dosage MTZ gels having suitable viscosities and other desirable properties utilizing carbomer 934, hydroxyethyl cellulose or polycarbophil as the gelling polymer are provided in Example 2.

As noted in the Summary, the solubility of MTZ presents problems when attempting to formulate MTZ in aqueous-based formulations, such as aqueous-based gels. Specific embodiments of the high dosage MTZ aqueous-based gels described herein utilize a novel solvent system which has been discovered to yield homogenous gels containing MTZ at concentrations as high as 2% by weight that are stable for long periods of time, for example at least about 6 months, when stored at temperatures ranging from about 25° C. to about 40° C. Surprisingly, such high dosage MTZ aqueous-based gels can be prepared without the aid of agents commonly used to enhance the solubility of MTZ in aqueous solutions, such as cyclodextrins, beta cyclodextrins, niacin, and/or niacinamide, and optionally also surfactants.

As used herein, “stable” means that the gel exhibits the following properties: (1) an MTZ purity of at least about 95% when stored at a temperature of about 25° C. for a period of at least 6 weeks; (2) no appreciable increase (e.g., no more than about a 2-fold to 3-fold increase) in formation of crystals and/or particulates upon microscopic visual inspection at a magnification of 40× when stored at a temperature of about 25° C. for a period of at least 6 weeks, as compared to a baseline value and (3) a change in viscosity of no more than about ±50% as measured at 25° C. when stored at a temperature of about 25° C. for a period of about 6 weeks, as compared to a baseline value or a control sample stored at a temperature in the range of about 2-8° C. In addition, it is desirable, but not required, that stable gels exhibit the following additional properties: (4) a change of pH of less than about ±0.3 pH units when stored at a temperature of about 25° C. for a period of 6 weeks, as compared to a baseline value or a control sample stored at a temperature in the range of about 2-8° C.; (5) a change in the efficacy of the preservative of no more than about ±10% when stored at about 25° C. for a period of at least about 6 weeks, as compared to a baseline value or a control sample stored at a temperature in the range of about 2-8° C. Assays suitable for measuring the stability of high dosage MTZ aqueous-based gels are provided in Example 3. Typically, the specific components and quantities of the novel solvent system are selected so as to yield a high dosage MTZ aqueous-based gel that is stable as defined herein. Guidance for selecting solvent systems useful for preparing stable high dosage MTZ gels having specific quantities of MTZ and other properties is provided by way of the various exemplary high dosage gels disclosed in the Examples section.

In some specific embodiments, the components and quantities of the novel solvent system are selected to yield a stable high dosage MTZ gel having the MTZ purity, homogeneity and viscosity properties discussed above, and optionally the pH and preservative efficacy properties discussed above, when stored at a temperature of about 40° C. for a period of about 6 weeks. In other specific embodiments, the components and quantities of the novel solvent system are selected to yield a stable high dosage MTZ gel having the above discussed MTZ purity, homogeneity and viscosity properties, and optionally the above-discussed pH and preservative efficacy properties, when stored at a temperature of in the range of about 25° C. to about 40° C., and in specific embodiments at a temperature of about 25° C. or about 40° C., for a period of 6 months, or even more, for example, for periods as long as 18 months. In yet other specific embodiments, the components and quantities of the novel solvent system are selected to yield a stable high dosage MTZ gel having the above-described MTZ purity, homogeneity, viscosity, pH and optionally preservative efficacy properties discussed above when stored at a temperature in the range of about 25° C. to about 40° C., and in specific embodiments at a temperature of about 25° C. or about 40° C., for a period of about 6 months, or even more, for example, for periods as long as about 18 months.

Various embodiments of novel solvent systems are described in the Summary section, supra. In some specific embodiments, the solvent system comprises about 2% to about 3% by weight of a first solvent having a saturated MTZ solubility at 25° C. of at least about 50 mg/g, and about 97% to about 98% by weight of one or more second solvents that collectively have a saturated MTZ solubility at 25° C. in the range of about 20 mg/g to about 25 mg/g. In a specific embodiment, each of the second solvents has a saturated MTZ solubility at 25° C. in the range of about 20 mg/g to about 25 mg/g. In some specific embodiments, the first solvent is an aromatic alcohol.

In some specific embodiments, the novel solvent system comprises one or more lower aromatic alcohol and one or more lower polyether, one or more lower polyol, and/or one or more polyoxyalkylene having a MW in the range of about 200 to about 600 (“lower polyoxyalkylenes”).

As used herein, a “lower aromatic alcohol” is an alcohol that has aromatic character and that contains from 6 to 15 carbon atom. In embodiments having greater than 6 carbon atoms, the lower aromatic alcohol may optionally include one or more heteroatoms replacing one or more carbon atoms. The aromatic character of the lower aromatic alcohol may be contributed by a pendant aromatic group (e.g., phenyl, naphthyl, etc.) on an alkyl or heteroalkyl chain that has aromatic character. Accordingly, the alcohol group of a lower aromatic alcohol may be phenolic (e.g., phenol), primary (e.g., benzyl alcohol), secondary (e.g., 2-methylbenzyl alcohol) or tertiary (e.g., 2-phenylpropane-2-ol) in character. Examples of lower aromatic alcohols include, but are not limited to, benzyl alcohol, 2-methylbenzyl alcohol and phenoxyethanol.

As used herein, a “lower polyether” is a straight-chained, branded or cyclic polyether containing from 2 to 15 backbone atoms, which are typically carbon and oxygen. The lower polyether may include additional functional groups, such as, for example, hydroxyl or oxy groups. Examples of lower polyethers include, but are not limited to, dialkyleneglycols (such, for example, diethylene glycol and diproylene glycol), alkyl ethers of isosorbide (such as, for example, dimethyl isosorbide), and tetraglycol.

Although carbonates are typically recognized as a distinct class of compounds, for purposes of the present application, carbonates are included within the definition of “lower polyethers.” Exemplary carbonate “lower polyethers” include, but are not limited to, carbonates of alkylene glycols (such as, for example, propylene carbonate).

As used herein, a “lower polyols” is a straight-chained, branded or cyclic compound containing two or more primary, secondary and/or tertiary alcohol groups. Lower polyols may include lower diols, such as lower aliphatic diols, defined below.

As used herein, “lower aliphatic diol” includes saturated or unsaturated, straight-chained, branched or cyclic aliphatic diols containing from two to ten carbon atoms. In some specific embodiments, the lower aliphatic diol is a saturated or unsaturated straight-chain or branched diol, referred to herein as a “lower alkylene diol” (also referred to as “lower glycols”). In some specific embodiments, the lower alkylene diol is a saturated straight-chained or branched diol, referred to herein as a “lower alkyl diol.” In some specific embodiments, the lower alkyl diol is a straight-chain diol, referred to herein as a “lower n-alkyl diol.” Specific examples of lower aliphatic diols useful in the solvent systems and gels described herein include, but are not limited to, ethane-1,2-diol (ethylene glycol), propane-1,2-diol (propylene glycol), propane-1,3-diol, butane-1,2-diol, butane-1,3-diol, butane-2,3-diol, butane-1,4-diol, pentane-1,5-diol, pentane-1,2-diol, 2-methyl-2,4-pentanediol, etc.

As used herein, “lower polyoxyalkylene” includes polymers formed from lower aliphatic diols and that have a molecular weight in the range of about 200 to about 600. Specific examples of lower polyoxyalkylenes useful in the solvent systems and gels described herein include, but are not limited to, polyethylene glycol (for example, PEG 200, PEG 400 and PEG 600), polypropylene glycol (for example PPG-9) and dipropylene glycol.

The solvent system may include additional agents and solvents, such as, for example, additional agents or solvents that enhance the solubility of MTZ in aqueous solutions, such as, for example, cyclodextrins, beta-cyclodextrins, niacin and/or niacinamide. Although solubility-enhancing agents can be used, it has been discovered that high dosage MTZ gels that are homogeneous and have good shelf stability properties can be prepared using solvent systems that do not include such additional solubilizing agents. Accordingly, in some specific embodiments, the solvent system and resultant gels are substantially free of agents that enhance the solubility of MTZ in aqueous solutions, such as cyclodextrins, beta-cyclodextrins, niacin, and/or niacinamide, and optionally also surfactants. In other specific embodiments, the solvent system is a ternary system that consists only of one or more lower aromatic alcohol(s), one or more lower aliphatic diol(s) and one or more lower polyoxyalkylene(s).

The solvent system will generally represent about 50% to about 80% by weight of the high dosage MTZ aqueous-based gel and in some specific embodiments, about 50% to about 70% by weight of the gel, or about 55% to about 60% by weight of the gel, although the gel may include higher or lower amounts of total solvents.

In some embodiments, solvent systems useful for solubilizing MTZ in the high dosage aqueous-based gels described herein comprise, relative to the resultant high dosage MTZ gel: (a) a polyoxyalkylene such as polyethylene glycol, preferably PEG 400, at a concentration of from about 10% to about 50%, by weight, for example from about 20% to about 40%, by weight, or from about 25% to about 39%, by weight, and in a specific embodiment about 37%, by weight; (b) a lower aromatic alcohol such as phenoxyethanol or benzyl alcohol, from about 1.1% to about 2% by weight, for example from about 1.3% to about 1.5% by weight, and in specific embodiments about 1.5% by weight; and/or (c) a lower aliphatic diol such as propane-1,2-diol, at a concentration of at least about 3% to about 20%, by weight, for example from about 15% to about 20% by weight and in a specific embodiment about 20% by weight.

The pH of the high dosage MTZ aqueous-based gels described herein should generally match the pH of the intended area of application and/or have a pH that is non-irritating upon application. For topical application to skin affected by rosacea, the pH should generally be in the range of about pH 5 to about pH 8. In some embodiments, the pH is in a range of about pH 5 to about pH 7. In still other embodiments, the pH is in a range of about pH 5.5 to about pH 6.5. The pH can be adjusted and/maintained with the aid of acids, bases buffers and other pH-adjusting agents, as is well-known in the art.

The high dosage MTZ aqueous-based gels described herein can also include other additional components, such as, for example, one or more preservatives, as is well-known in the art. When used, preservative(s) should generally represent no more than about 1% or 2% by weight of the high dosage MTZ aqueous-based gel. The choice of preservative(s) is not critical. Preservatives suitable for use in topical pharmaceuticals are well-known to those of skill. In some specific embodiments the one or more preservative(s) are esters of 4-hydroxy benzoic acid, also known as parabens. Suitable parabens include lower alkyl esters of 4-hydroxy benzoic acid, such as, for example, methyl 4-hydroxybenzoate (methyl parben), ethyl 4-hydroxybenzoate (ethyl paraben) and propyl 4-hydroxy-benzoate (propyl paraben).

Skilled artisans will appreciate that solvents used to solubilize the MTZ in the high dosage MTZ aqueous-based gels described herein may also have preservative properties. For example, benzyl alcohol has well known preservative properties. When used as a solvent in the novel solvent system, the preservative properties can be used to advantage. Indeed, gels including solvents with preservative properties need not necessarily include additional preservatives. Gels that utilize the preservative properties of solvents included in the solvent system should, in cases where the solvent may degrade or oxidize over time, include an amount of overage that takes into account the degradation and/or oxidation such that the gel retains an amount of undegraded and/or unoxidized solvent that, in addition to solubilizing the MTZ, has effective preservative properties after a desired period of time. For example, benzyl alcohol is known to oxidize to benzaldehyde, which does not have preservative properties. In embodiments of the high dosage MTZ gels described herein that employ benzyl alcohol as an MTZ solvent and as a preservative, an amount of benzyl alcohol should be included in the gel that not only solubilizes the MTZ, but that yields a preservative effect for the duration of the expected shelf life of the gel. Overage amounts of benzyl alcohol, or other solvents employed in the solvent system as preservatives, can be determined based upon the degradation properties and kinetics of the particular solvent under the desired conditions of storage.

Embodiments of high dosage MTZ aqueous-based gels that include solvents having preservative properties may also include one or more additional preservatives, and/or preservatives designed to protect the solvent from degradation. For example, in the case of benzyl alcohol, the high dosage gels described herein may include one or more additional preservatives that have antioxidant properties, in part to protect the benzyl alcohol from oxidation. In a specific embodiment, high dosage MTZ aqueous-based gels comprising benzyl alcohol or other solvents that oxidize include one or more parabens as additional preservatives. In some specific embodiments the high dosage MTZ aqueous-based gels comprise about 0.1% by weight total parabens, and in some specific embodiments about 0.02% by weight methyl paraben and about 0.08% by weight propyl paraben.

The high dosage MTZ aqueous-based gels also include water, either in the form of pure water, or in the form of an aqueous buffer. Typically, the amount of water included in the gel will be less than about 70% by weight of the gel, more typically less than about 60% by weight, in some specific embodiments in the range of about 30% to about 40% by weight, and in still further specific embodiments about 35% to about 45% by weight of the gel.

A specific exemplary high dosage MTZ aqueous-based gel comprises:

(a) about 1.5% MTZ by weight; and

(b) one or more gelling polymers.

Another specific exemplary high dosage MTZ aqueous-based gel comprises:

(a) about 1.5% MTZ by weight;

(b) one or more gelling polymers;

(c) about 1.5% by weight benzyl alcohol or phenoxyethanol; and

(d) about 57% by weight of one or more solvents having a saturated MTZ solubility at 25° C. of at least about 20 mg/g.

Another specific exemplary high dosage MTZ aqueous-based gel comprises:

(a) about 1.5% MTZ by weight MTZ;

(b) about 1.5% by weight benzyl alcohol or a solvent having a saturated MTZ solubility at 25° C. of at least about 50 mg/g; and

(c) about 50% by weight to about 60% by weight of one or more solvents collectively having a saturated MTZ solubility at 25° C. in the range of about 20 mg/g to about 25 mg/g.

Another specific exemplary high dosage MTZ aqueous-based gel comprises:

(a) about 1.5% by weight MTZ;

(b) about 1% to about 2% by weight, and in some specific embodiments about 1.5% by weight, of one or more aromatic alcohols; and

(c) about 50% to about 60% by weight, and in some specific embodiments about 57% by weight of one or more solvents selected from the group consisting of propane-1,2-diol, propylene carbonate, dipropylene glycol, an isosorbide dialkyl ether, dimethyl isosorbide (Arlasolve®), PEG 400, and PEG 200.

Another specific exemplary high dosage MTZ aqueous-based gel comprises:

(a) about 1.5% by weight MTZ;

(b) about 1% to about 2% by weight, and in some specific embodiments about 1.5% by weight, of one or more aromatic alcohols;

(c) about 30% to about 40% by weight, and in some specific embodiments about 37% by weight, of one or more polyoxyalkylenes; and

(d) about 20% to about 25% by weight, and in some specific embodiments about 20% by weight, of one or more lower polyols, lower aliphatic diols and/or lower polyethers.

Another specific exemplary high dosage MTZ aqueous-based gel comprises:

(a) about 1.5% by weight MTZ;

(b) about 1.5% by weight aromatic alcohols, for example, benzyl alcohol or phenoxyethanol;

(c) about 37% by weight of total lower polyoxyalkylenes, for example PEG 400, PEG 200 or a mixture thereof; and

(d) about 20% by weight of one or more lower diols (for example dipropylene glycol and/or propane-1,2-diol) one or more lower polyethers (for example, dimethyl isosorbide and/or propylene carbonate).

Another specific embodiment of an exemplary high dosage MTZ gel is a gel according to any of the above-described specific embodiments that further includes one or more of the following:

(a) about 1% by weight to about 2% by weight, and in some embodiments about 1% by weight, of one or more gelling polymers, optionally selected from a cross-linked polymer of acrylic acid (for example a carbomer) and a cellulosic polymer (for example HEC);

(b) about 35% by weight to about 40% by weight, and in some specific embodiments about 37.9% by weight, of water; and

(c) about 0.1% by weight of one or more preservatives, and in some specific embodiments a mixture of parabens, for example a 1:4 wt/wt mixture of propylparaben and methylparaben.

Still another specific embodiment of an exemplary high dosage MTZ aqueous-based gel includes any of the above-described embodiments which are substantially free of cyclodextrins, beta-cyclodextrins, niacin, and niacinamide, and optionally also surfactants.

Yet another specific embodiment of an exemplary high dosage MTZ aqueous-based gel is a gel, and as a specific example any of the above-described exemplary specific embodiments, in which the solvent system is a mixture of solvents defined by one of systems 1-18, provided in Tables A and B below:

TABLE A
Exemplary Solvent Systems

| | 1 | 2 | 3 | 4 | 5 | 6 | 7 | 8 | 9 |
|---|---|---|---|---|---|---|---|---|---|
| Benzyl alcohol | 1.50 | 1.50 | 1.50 | 1.50 | 1.50 | 1.50 | 1.50 | 1.50 | 1.50 |
| Phenoxy ethanol | — | — | — | — | — | — | — | — | — |
| Propylene glycol | — | 13.00 | — | 20.00 | — | 20.00 | — | 13.00 | 20.00 |
| Propylene carbonate | 5.00 | 5.00 | 5.00 | 5.00 | 5.00 | 5.00 | — | — | 2.00 |
| Dipropylene glycol | — | — | 5.00 | 5.00 | — | — | 5.00 | 5.00 | — |
| Arlosolve | — | — | 15.00 | 15.00 | 15.00 | 15.00 | — | — | 15.00 |
| PEG 400 | 13.00 | — | 32.00 | 12.00 | 37.00 | 17.00 | 13.00 | — | — |
| PEG 200 | 39.00 | 39.00 | — | — | — | — | 39.00 | 39.00 | 20.00 |

TABLE B
Exemplary Solvent Systems

| | 10 | 11 | 12 | 13 | 14 | 15 | 16 | 17 | 18 |
|---|---|---|---|---|---|---|---|---|---|
| Benzyl alcohol | 1.50 | 1.50 | 1.50 | 1.50 | 1.50 | 1.50 | 1.50 | — | 1.50 |
| Phenoxy ethanol | — | — | — | — | — | — | — | 1.50 | — |
| Propylene glycol | 20.00 | — | 20.00 | — | 18.00 | 5.00 | 20.00 | 20.00 | 20.00 |
| Propylene carbonate | — | — | — | — | — | — | — | — | — |
| Dipropylene glycol | — | 5.00 | 5.00 | — | — | — | — | — | — |
| Arlosolve | — | 15.00 | 15.00 | — | — | 15.00 | 15.00 | — | — |
| PEG 400 | 17.00 | 37.00 | 17.00 | 18.00 | — | 37.00 | 22.00 | 37.00 | 37.00 |
| PEG 200 | 20.00 | — | — | 39.00 | 39.00 | — | — | — | — |

### 5.3. Methods of Making Gels

The high dosage MTZ aqueous-based gels described herein may generally be prepared by dissolving the water-soluble components of the gel in water or buffer to yield an aqueous solution, mixing the components of the MTZ solvent system and dissolving the desired quantity of MTZ in the MTZ solvent system to yield and MTZ solution, mixing together the required amounts of the aqueous solution and MTZ solution, adjusting the pH if necessary, and adding the desired quantity of gelling agent to the mixture. The pH of the resultant gel may be adjusted with acid or base to within a desired range. Specific methods for making high dosage MTZ aqueous-based gels are provided in Example 2.

### 5.4. Uses

The high dosage MTZ aqueous-based gels described herein can be used to topically administer MTZ in any context where such administration would be beneficial. In specific embodiments described further below, the gels can be used topically to treat subjects suffering from and/or diagnosed with rosacea.

Rosacea is a chronic skin disease that can affect the cheeks, nose, eyes, chin, and forehead. It is characterized by recurrent episodes of flushing (redness), papules (pimples), pustules, and telangiectasias (permanent distended blood capillaries with a spidery appearance). See, e.g., Crawford 2004; Korting 2009; Powell 2005. Although there is no standard clinical definition of the condition, rosacea is generally classified into four subtypes and one variant ((see, e.g., Wilkin et al., 2002, “Standard Classification of Rosacea: Report of the National Rosacea Society Expert Committee on the Classification and Staging of Rosacea,” J Am Acad Dermatol 46(4):584-587 (“Wilkin 2002”); Wilkin et al., 2004, Standard Classification of Rosacea: Report of the National Rosacea Society Expert Committee on the Classification and Staging of Rosacea,” J Am Adad Dermatol 50(6):907-912 (“Wilkin 2004”)):
Subtype 1—Erythematotelangiectatic rosacea. Clinical features include flushing and persistent central facial erythema (redness) with or without telangiectasias.
Subtype 2—Papulopustular Rosacea. Characterized by persistent central facial erythema with transient, central face papules or pustules, or both.
Subtype 3—Phymatous rosacea. Characterized by thickening of the skin with irregular surface nodalities, and enlargement. This may occur on the nose (rhinophyma), chin, forehead, cheeks, or ears.
Subtype 4—Ocular rosacea. Characterized by ocular involvement, including inflammation of different parts of the eye and eyelid.
Variant—Granulomatous rosacea. Non-inflammatory variant characterized by hard brown, yellow, or red cutaneous papules, or nodules of uniform size.

Individuals manifesting one or more of the symptoms noted above for the various rosacea subtypes are considered suffering from and/or diagnosed with rosacea. In some specific embodiments, individuals are considered to be suffering from and/or diagnosed with rosacea who exhibit the inclusion criteria for clinical studies of MTZ for the treatment of rosacea delineated in an FDA “Draft Guidance on Metronidazole,” dated March 2010, the content of which is incorporated herein by reference. These inclusion criteria establish a clinical diagnosis of moderate to severe facial rosacea and include:
a total of 8 to 50 combined papules/pustules on the face;
at least moderate erythema; and
telangiectasia.

The methods generally involve applying to the affected area of the skin an amount of a high dosage MTZ aqueous-based gel as described herein for a number of applications sufficient to provide a therapeutic benefit. As used herein, a “therapeutic benefit” is achieved when one or more of the symptoms of rosacea and/or one or more of the clinical manifestations of rosacea, are ameliorated, eliminated, eradicated or improved. A treatment regimen can provide therapeutic benefit to a subject without curing the underlying rosacea condition.

As used herein, a symptom or manifestation of rosacea is ameliorated if it is decreased in magnitude after therapy, or moves closer to a level considered normal. For example, a reduction in the severity of erythema, or a reduction in the number of lesions (pustules and/or papules) illustrate amelioration of symptoms. Symptoms of rosacea are considered eliminated or eradicated if the symptoms are no longer detectable using well-known detection means.

Therapeutic benefit may also be defined by reference to established primary and/or secondary outcomes, for example:
improvement on quality of life;
patient-assessed improvement in rosacea severity;
physician-assessed and/or patient-assessed improvement in rosacea severity, including
   ≧50% improvement in global evaluation
   ≧50% reduction in lesion counts
   reduction in time needed for improvement of skin lesions
   increased duration of periods of remission.

Other criteria for establishing therapeutic benefit may be found in the Draft FDA Guidance on Metronidazole, for example, at page 2, ¶7.

As used herein, a “therapeutically effective amount” refers to a treatment regimen with a high dosage MTZ aqueous-based gel that provides therapeutic benefit, or that when applied prophylactically (for example, between rosacea outbreaks), delays the onset of one or more symptoms or clinical manifestations of rosacea.

The frequency and duration of application can vary, and may depend upon the desired outcome. As discussed above, the high dosage MTZ aqueous-based gels described herein deliver unusually high quantities of MTZ to the stratum corneum, which is believed may act as a reservoir for the MTZ and may permit less frequent application than current gels and/or creams and/or a shorter overall duration of therapy.

Accordingly, in some embodiments, it is expected that an amount of high dosage MTZ aqueous-based gel that contains from about 3 mg to about 10 mg MTZ applied to the affected area about the size of an adult palm or cheek (e.g., approx. 64 cm²) once or twice per day for a period of up to about 12 weeks, and even for periods as short as 11 weeks, 10 weeks, 9 weeks, 8 weeks, 7 weeks, or 6 weeks, or 5 weeks, or even 4 weeks, is expected to provide therapeutic benefit. In some specific embodiments, the amount of gel applied contains about 3.75 mg MTZ.

Owing to the expected reservoir or depot effect of the gels described herein, it is expected that less frequent applications may also yield therapeutic benefit, for example, once daily, once every other day, or even once every third day.

It is also possible that the high dosage MTZ aqueous-based gels described herein may also provide therapeutic benefit by increasing the length of asymptomatic periods or periods of remission from symptoms between outbreaks. In some specific embodiments, the high dosage MTZ aqueous-based gels described herein may be applied for a first treatment regimen, such as one of the treatment regimens described above, treatment then discontinued for a period of time, and a treatment regimen resumed either when symptoms reappear or before symptoms reappear, for example, when exposure to conditions that trigger rosacea outbreaks are expected to be experienced. The length of time that treatment is discontinued may range from a period of days, to weeks, to a month, to a few months, to several months, depending upon the individual and the circumstances.

For use, the high dosage MTZ aqueous-based gels can be packaged in any form that is convenient for the desired mode of application. In specific embodiments, the high dosage MTZ aqueous-based gels are packaged in unit dosage form, for example in tubes or other packages that contain an amount of gel suitable for a single application.

### 5.5. Additional Non-Limiting Aspects

Additional non-limiting aspects of the high dosage MTZ aqueous-based gel compositions and methods of using them to treat individuals suffering from and/or diagnosed with rosacea are provided below.

5.5.1. Methods

M1. A method of treating a subject suffering from and/or diagnosed with rosacea, comprising topically applying to the affected area an amount of a high dosage MTZ gel at a frequency and for a total number of applications sufficient to provide therapeutic benefit, where the high dosage MTZ gel comprises about 1.25% to about 2% by weight MTZ, one or more gelling polymers, and water.

M2. The method of aspect M1, wherein the levels of MTZ from the high dosage MTZ gel measured in the stratum corneum and receiver fluid in an in vitro Franz cell skin permeation experiment carried out with ¹⁴C-labeled MTZ and human cadaver skin are at least about 25 to 55-fold higher and at least about 1 to 20-fold lower, respectively, when normalized for concentration, than the MTZ levels measured from 0.75% METROGEL®.

M3. The method of any one of aspects M1-M2, in which the high dosage MTZ gel has a viscosity ranging from about 30,000 mPa to about 500,000 mPa, measured at 25° C. using the controlled shear rate ramp method, a Bohlin CVO 100 rheometer and the rheometer parameters of Table 7.

M4. The method of aspect M3, in which the high dosage MTZ gel has viscosity in the range of about 75,000 mPa to about 125,000 mPa.

M5. The method of any one of aspects M1-M4, in which the high dosage MTZ gel comprises about 35 wt % to about 40 wt % water, and in some specific aspects about 37.9% by weight water.

M6. The method of any one of aspects M1-M5, in which the high dosage MTZ gel is substantially free of dextrins, cyclodextrins, niacin, and niacinamide, and optionally also surfactants.

M7. The method of any one of aspects M1-M6 in which the high dosage MTZ gel is stable for a period of at least 6 months at 25° C.

M8. The method of any one of aspects M1-M7 in which the high dosage MTZ gel includes about 1 wt % to about 3 wt % total gelling polymers and about 45 wt % to about 65 wt % of a solvent system for the MTZ, and in some specific aspects about 58.5 wt % of a solvent system for the MTZ.

M9. The method of aspect M8 in which the solvent system comprises one or more solvents having a saturated MTZ solubility at 25° C. of at least about 20 mg/g.

M10. The method of aspect M9 in which the solvent system comprises one or more first solvents having a saturated MTZ solubility at 25° C. of at least about 50 mg/g and optionally one or more second solvents having a saturated MTZ solubility at 25° C. in a range of about 20 mg/g to about 25 mg/g.

M11. The method of aspect M10 in which the solvent system comprises one or more lower aromatic alcohols, and optionally one or more lower aliphatic diols, one or more lower polyethers and/or one or more polyoxyalkylenes having a molecular weight ranging from about 200 to about 400 (“lower polyoxyalkylene”).

M12. The method of aspect M11 in which the solvent system comprises one or more lower aromatic alcohols, one or more lower aliphatic diols, and one or more lower polyoxyalkylenes.

M13. The method of aspect M11 in which the solvent system comprises one or more lower aromatic alcohols, one or more lower polyethers, and one or more lower polyoxyalkylenes.

M14. The method of any one of aspects M11-M13 in which the one or more lower aromatic alcohols are selected from the group consisting of benzyl alcohol, 2-methylbenzyl alcohol, phenoxyethanol, and mixtures thereof.

M15. The method of any one of aspects M11-M14 in which the one or more lower aliphatic diols are selected from the group consisting of ethane-1,2-diol (ethylene glycol), propane-1,2-diol (propylene glycol), and mixtures thereof, and the one or more lower polyethers are selected from the group consisting of propylene carbonate, dipropylene glycol, an isosorbide alkyl ether, dimethyl isosorbide, and tetraglycol.

M16. The method of any one of aspects M11-M15 in which the one or more lower polyoxyalkylenes are selected from the group consisting of polyoxyethylene (polyethylene glycol), polyoxypropylene (polypropylene glycol), and mixtures thereof.

M17. The method of any one of aspects M11-M16 in which the one or more lower polyoxyethylenes are selected from the group consisting of PEG 400 and PEG 200.

M18. The method of aspect M10 in which the solvent system comprises about 2 wt % to about 5 wt % of the first solvent(s) and about 95 wt % to about 98 wt % of the second solvent(s).

M19. The method of aspect M18 in which the first solvent is benzyl alcohol or phenoxyethanol and the second solvents are each selected from the group consisting of propane-1,2-diol, propylene carbonate, dipropylene glycol, dimethyl isosorbide, PEG 200, and PEG 400.

M20. The method of any one of aspects M8-M19 in which the solvent system consists of benzyl alcohol and/or phenoxyethanol, and one or more of propane-1,2-diol, propylene glycol, propylene carbonate, dipropylene glycol, dimethyl isosorbide, PEG 400, and PEG 200.

M21. The method of any one of aspects M8-M20 in which the solvent system consists of a solvent system selected from Table A or Table B.

M22. The method of any one of aspects M1-M21 in which the high dosage MTZ gel further comprises one or more preservatives.

M23. The method of any one of aspects M1-M22 in which the one or more preservative is an ester of 4-hydroxy benzoic acid (a paraben).

M24. The method of aspect M23 in which the one or more preservatives are selected from the group consisting of methyl 4-hydroxybenzoate (methylparaben), propyl 4-hydroxybenzoate (propylparaben), and mixtures thereof.

M25. The method of any one of aspects M1-M23 in which the high dosage MTZ gel has a pH in the range about pH 5 to about pH 8.

M26. The method of aspect M25 in which the high dosage MTZ gel has a pH in the range of about pH 5.5 to about pH 6.5.

M27. The method of any one of aspects M1-M26 in which the one or more gelling polymer is selected from the group consisting of a cross-linked acrylic acid polymer, a cellulosic polymer and mixtures thereof, and in some specific aspects the gelling polymer is a carbomer.

M28. The method of any one of aspects M1-M27 in which the amount of high dosage MTZ gel applied in a single application contains about 3.75 mg MTZ.

M29. The method of any one of aspects M1-M28 in which the high dosage MTZ gel includes about 1.5 wt % MTZ.

M30. The method of aspect M29 in which the high dosage MTZ gel is applied once or twice per day for a period of 4 to 12 weeks.

M31. The method of aspect M30 in which the high dosage MTZ gel is applied once per day.

M32. The method of aspect M29 in which the high dosage MTZ gel is applied every other day for a period of 4 to 12 weeks.

M33. The method of aspect M29 in which the high dosage MTZ gel is applied twice weekly for a period of 4 to 12 weeks.

M34. The method of aspect M33 in which the high dosage MTZ gel is applied once weekly for a period of 4 to 12 weeks.

M35. The method of any one of aspects M1-M36 in which the high dosage MTZ gel is selected from the group consisting of F17, F17_{pH6}, F17_{pH8}, F20_{pH6} and F20_{pH8}.

M36. The method of any one of aspects M-1-M36 in which the high dosage MTZ gel is selected from the group consisting of any one of gels V1-V17.

M37. The method of any one of aspects M-1-M36 in which the high dosage MTZ gel is F17.

M38. The method of any one of aspects M35-M37 in which the high dosage MTZ gel is applied once or twice per day for a period of 4 to 12 weeks, and in some specific aspects for a period of 4, 5, 6, 7, 8, 9 or 10 weeks.

M39. The method of any one of aspects M35-M37 in which the high dosage MTZ gel is applied every other day for a period of 4 to 12 weeks, and in some specific aspects for a period of 4, 5, 6, 7, 8, 9 or 10 weeks.

M40. The method of any one of aspects M35-M37 in which the high dosage MTZ gel is applied once or twice weekly for a period of 4 to 12 weeks, and in some specific aspects for a period of 4, 5, 6, 7, 8, 9 or 10 weeks.

M41. The method of any one of aspects M35-M40 in which the amount of high dosage MTZ gel applied in a single application contains about 3.75 mg MTZ.

M42. A method of treating a subject suffering from and/or diagnosed with rosacea, comprising applying topically to the affected area an amount of a high dosage MTZ gel sufficient to provide therapeutic benefit, wherein the high dosage MTZ gel comprises one or more gelling polymers, about 1.5 wt % MTZ, and water, and has one or more features or characteristics selected from the following group:

(a) the levels of MTZ from the high dosage MTZ gel measured in the stratum corneum and receiver fluid in an in vitro Franz cell skin permeation experiment carried out with ¹⁴C-labeled MTZ and human cadaver skin are at least about 25 to 55-fold higher and at least about 1 to 20-fold lower, respectively, when normalized for concentration, than the MTZ levels measured from 0.75% METROGEL®;

(b) a viscosity ranging from about 60,000 mPa to about 500,000 mPa, measured at 25 C using the controlled shear rate ramp method, a Bohlin CVO 100 rheometer and the rheometer parameters of Table 7.

(c) comprises about 30 wt % to about 60 wt % water;

(d) is substantially free of dextrins, cyclodextrins, niacin, and niacinamide, and optionally also surfactants;

(e) is stable for a period of at least 6 months at 25° C.;

(f) includes about 1 wt % to about 3 wt % total gelling polymers; and

(g) includes about 55% by weight to about 60% by weight, and in some specific aspects about 58.5% by weight, of a solvent system for the MTZ.

M43. The method of aspect M42 in which the high dosage MTZ gel is applied once or twice daily for a period of 4 to 12 weeks.

M44. The method of any one of aspects M1-M43 where the high dosage MTZ gel is applied for a first course of treatment, treatment discontinued for a period of time, and the treatment resumed for at least a second course of treatment.

M45. The method of aspect M44 in which the treatment is discontinued for a period of days, weeks, one month, two months, three months, or longer.

5.5.2. Compositions

C1. A high dosage MTZ gel comprising about 1 wt % to about 2 wt % MTZ, about 1 wt % to about 3 wt % of one or more gelling polymers, about 30 wt % to about 60 wt % of a solvent system for the MTZ, and water, where the levels of MTZ from the high dosage MTZ gel measured in the stratum corneum and receiver fluid in an in vitro Franz cell skin permeation experiment carried out with ¹⁴C-labeled MTZ and human cadaver skin are at least about 25 to 55-fold higher and at least about 1 to 20-fold lower, respectively, when normalized for concentration, than the MTZ levels measured from 0.75% METROGEL®.

C2. A high dosage MTZ gel comprising about 1 wt % to about 2 wt % MTZ, about 1 wt % to about 3 wt % of one or more gelling polymers, about 30 wt % to about 60 wt % of a solvent system for the MTZ, and water, wherein the high dosage MTZ gel has a viscosity ranging from about 60,000 mPa to about 500,000 mPa, measured at 25° C. using the controlled shear ramp method, a Bohlin CVO 100 rheometer and the rheometer parameters of Table 7.

C3. The gel of aspect C2 which has a viscosity in a range of about 75,000 mP to about 125,000 mPa.

C4. A high dosage MTZ gel comprising about 1 wt % to about 2 wt % MTZ, about 1 wt % to about 3 wt % of one or more gelling polymers, about 30 wt % to about 60 wt % of a solvent system for the MTZ, and about 35 wt % to about 40 wt % water, and in some specific aspects about 37.9 wt % water.

C5. A high dosage MTZ gel comprising about 1 wt % to about 2 wt % MTZ, about 1 wt % to about 3 wt % of one or more gelling polymers, about 30 wt % to about 60 wt % of a solvent system for the MTZ, and water, wherein the high dosage MTZ gel is substantially free of dextrins, cyclodextrins, niacin and niacinamide, and optionally also surfactants.

C6. A high dosage MTZ gel comprising about 1 wt % to about 2 wt % MTZ, about 1 wt % to about 3 wt % of one or more gelling polymers, about 30 wt % to about 60 wt % of a solvent system for the MTZ, and water, wherein the high dosage MTZ gel is stable for a period of at least 6 months at 25° C.

C7. The gel of any one of aspects C1-C6 in which the high dosage MTZ gel includes about 1 wt % to about 3 wt % total gelling polymer and about 55-65 wt % of a solvent system for the MTZ.

C8. The gel of aspect C7 in which the solvent system comprises one or more solvents having a saturated MTZ solubility at 25° C. of at least about 20 mg/g.

C9. The gel of aspect C8 in which the solvent system comprises one or more first solvents having a saturated MTZ solubility at 25° C. of at least about 50 mg/g and optionally one or more second solvents having a saturated MTZ solubility at 25° C. in a range of about 20 mg/g to about 25 mg/g.

C10. The gel of aspect C9 in which the solvent system comprises one or more lower aromatic alcohols, and optionally one or more lower aliphatic diols, one or more lower polyethers and/or one or more polyoxyalkylenes having a molecular weight ranging from about 200 to about 400 (“lower polyoxyalkylene”).

C11. The gel of aspect C10 in which the solvent system comprises one or more lower aromatic alcohols, one or more lower aliphatic diols, and one or more lower polyoxyalkylenes.

C12. The gel of aspect C10 in which the solvent system comprises one or more lower aromatic alcohols, one or more lower polyethers, and one or more lower polyoxyalkylenes.

C13. The gel of any one of aspects C10-C12 in which the one or more lower aromatic alcohols are selected from the group consisting of benzyl alcohol, 2-methylbenzyl alcohol, phenoxyethanol, and mixtures thereof.

C14. The gel of any one of aspects C10-C13 in which the one or more lower aliphatic diols are selected from the group consisting of ethane-1,2-diol (ethylene glycol), propane-1,2-diol (propylene glycol), and mixtures thereof, and the one or more lower polyethers are selected from the group consisting of propylene carbonate, dipropylene glycol, an isosorbide alkyl ether, dimethyl isosorbide, and tetraglycol.

C15. The gel of any one of aspects C10-C14 in which the one or more lower polyoxyalkylenes are selected from the group consisting of polyoxyethylene (polyethylene glycol), polyoxypropylene (polypropylene glycol), and mixtures thereof.

C16. The gel of any one of aspects C10-C15 in which the one or more lower polyoxyalkylenes are selected from the group consisting of PEG 400, PEG 200, and mixtures thereof.

C17. The gel of any one of aspects C10-C16 in which the solvent system comprises about 3.5 wt % to about 5 wt % of the first solvent(s) and about 95 wt % to about 98 wt % of the second solvent(s).

C18. The gel of aspect C17 in which the first solvent is benzyl alcohol or phenoxyethanol and the second solvents are each selected from the group consisting of propane-1,2-diol, propylene carbonate, dipropylene glycol, dimethyl isosorbide, PEG 200, and PEG 400.

C19. The gel of any one of aspects C10-C18 in which the solvent system consists of benzyl alcohol and/or phenoxyethanol, and one or more of propane-1,2-diol, propylene glycol, propylene carbonate, dipropylene glycol, dimethyl isosorbide, PEG 400, and PEG 200.

C20. The gel of any one of aspects C10-C19 in which the solvent system consists of a solvent selected from Table A or Table B.

C21. The gel of any one of aspects C1-C20 which further comprises one or more preservatives.

C22. The gel of any one of aspects C1-C21 in which the one or more preservative is an ester of 4-hydroxy benzoic acid (a paraben).

C23. The gel of aspect C22 in which the one or more preservatives are selected from the group consisting of methyl 4-hydroxybenzoate (methylparaben), propyl 4-hydroxybenzoate (propylparaben), and mixtures thereof.

C24. The gel of any one of aspects C1-C23 which has a pH in a range about pH 5 to about pH 8.

C25. The gel of aspect C24 which has a pH in a range of about pH 5.5 to about pH 6.5.

C26. The gel of any one of aspects C1-C25 which includes about 1.5% by weight MTZ.

C27. The gel of any one of aspects C1-C26 which includes about 1% or 2% by weight total gelling polymer.

C28. The gel of aspect C27 in which the one or more gelling polymer is selected from the group consisting of a cross-linked acrylic acid polymer and a cellulosic polymer and mixtures thereof.

C29. The gel of aspect C28 in which the gelling polymer is a carbomer.

5.5.3. Uses

U1. A use of a high dosage MTZ gel according to any one of aspects C1-C28 for the treatment of a subject suffering from and/or diagnosed with rosacea.

U2. The use of aspect U1 in which the gel is applied once or twice daily for a period of time sufficient to provide therapeutic relief.

U3. The use of any one of the aspects U1-U2 in which the gel is applied once daily for a period of 4 to 12 weeks.

U4. The use of aspect U3 in which the gel is applied once every other day for a period of 4 to 12 weeks.

U5. The use of aspect U3 in which the gel is applied once every third day for a period of 4 to 12 weeks.

U6. The use of aspect U3 in which the gel is applied once a week for a period of 4 to 12 weeks.

U7. The use of any one of aspects U3-U6 in which the gel is applied for 6, 7, 8, 9 or 10 weeks.

U8. The use of any one of aspects U1-U7 in which the amount of gel is applied in a single application that contains about 3 mg to about 10 mg MTZ.

U9. The use of any one of aspects U1-U7 in which the amount of gel applied in a single application contains about 3.75 mg MTZ.

U10. The use of any one of aspects U1-U9 in which a first course of treatment is applied, treatment discontinued for a period of time, and then resumed for at least a second course of treatment.

U11. The use of aspect U10 in which the period of time the treatment is discontinued is days, weeks, several weeks, a month, two months, three months or even longer.

5.5.4. Unit Dosage Forms

D1. A unit dosage form of a high dosage MTZ gel suitable for topical application, comprising an amount of a high dosage MTZ gel according to any one of aspects C1-C28 suitable for a single application packaged in a container suitable for dispensing the gel.

D2. The unit dosage form of aspect D1 which contains an amount of gel containing about 3 mg to about 10 mg MTZ.

D3. The unit dosage form of aspect D2 in which the container contains about 3.75 mg MTZ.

Unless otherwise defined, all technical and scientific terms used herein have the same meaning as commonly understood by one of ordinary skill in the art to which the various inventions described herein belong. Although methods and materials similar or equivalent to those described herein can be used in the practice or testing of the disclosed inventions, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### 6. EXAMPLES

Having now generally described the invention, the same will be more readily understood through reference to the following Examples which are provided by way of illustration, and are not intended to be limiting of the present invention, unless specified.

The following examples are put forth for illustrative purposes only and are not intended to limit the scope of what the inventors regard as their invention.

### Example 1

### Exemplary Solvents Useful for Solvent Systems

The solubility of MTZ in a variety of solvents at 25° C. was determined to identify solvents useful for solubilizing MTZ at the concentrations required for the high dosage MTZ aqueous-based gels described herein. The saturated MTZ solubility at 25° C. of a number of solvents are provided in Table 1, below. Solvent systems useful for preparing high dosage mucoadhesive MTZ aqueous-based gels having desired concentrations of MTZ can be devised using these saturated solubilities for guidance. Additional solvents suitable for use with these (and other) solvents for preparing high dosage mucoadhesive MTZ aqueous-based gels as described herein may be readily identified based upon their saturated MTZ solubilities. The saturated MTZ solubility at 25° C. in a number of solvent systems that have been mixed with an aqueous phase that are suitable for preparing high dosage MTZ aqueous-based gels are provided in Table 2, below.

Additional guidance may be obtained from the solvent systems used to prepare the exemplary MG, F, and V-series high dosage MTZ aqueous-based gels provided in Example 2.

TABLE 1

| | Solubility at | |
|---|---|---|
| | 25° C. (mg/g) | Solubility at |
| | (n = 3, | 25° C. (mg/g) |
| | mean ± SEM) | (n = 1) |
| Benzyl alcohol | | 72.69 |
| 2 wt % aq. Benzyl alcohol | 10.81 ± 0.040 | |
| Deionized water | 8.70 ± 0.09 | 9.37 |
| Ethanol | 16.36 ± 0.12 | 19.96 |
| Ethylene glycol | | 21.34 |
| Glycerol | | 8.35 |
| 50 wt % aq. glycerol | 7.62 ± 0.19 | |
| Propylene glycol | 18.62 ± 0.25 | 20.74 |
| PEG 400 | 18.79 ± 1.23 | 24.78 |
| Phosphate buffer, 50 mM, pH 4 | 8.26 ± 0.45 | 9.09 |
| Phosphate buffer, 50 mM, pH 5 | | 9.13 |
| Phosphate buffer, 50 mM, pH 6 | 8.57 ± 0.05 | 9.10 |
| Phosphate buffer, 50 mM, pH 7 | | 9.07 |
| tetraglycol | | 29.65 |
| 10 wt % aq. Lutrol ® F127 | 9.15 ± 0.09 | |
| 1 wt % aq. polysorbate 60 | 9.72 ± 0.08 | |

TABLE 2
Saturated MTZ Solubility at 25° C. of Solvent Mixtures

| | 3 | 4 | 5 | 6 | 7 | 18 | 19 | 20 | 21 | 22 | 23 | 24 |
|---|---|---|---|---|---|---|---|---|---|---|---|---|
| Benzyl alcohol | 5.00 | — | — | 2.00 | — | 1.50 | 1.50 | 1.50 | 1.50 | 1.00 | 1.00 | 1.00 |
| Propylene glycol | 20.00 | 20.00 | 20.00 | 15.00 | — | 10.00 | 20.00 | 20.00 | 20.00 | 20.00 | 20.00 | 20.00 |
| PEG 400 | 20.00 | 20.00 | 20.00 | 25.00 | — | 20.00 | 20.00 | 20.00 | 20.00 | 20.00 | 20.00 | 20.00 |
| ethanol | — | 10.00 | 20.00 | — | — | — | — | — | — | — | — | — |
| Polysorbate 60 | — | — | — | — | — | — | — | 1.00 | — | — | — | — |
| Lutrol ® F127 | — | — | — | — | — | — | — | — | 10.00 | — | — | 10.00 |
| glycerol | — | — | — | — | — | — | — | — | — | — | — | — |
| β-HPC | — | — | — | — | 20.00 | — | — | — | — | — | — | — |
| Deionized water | — | — | — | — | 8.00 | — | — | — | — | — | — | — |
| Phosphate buffer, 50 mM, pH 4 | 55.00 | 50.00 | 40.00 | 58.00 | — | 68.50 | 58.50 | 57.50 | | 59.00 | 58.00 | 49.00 |
| MTZ solubility (% w/w) | 1.56 | 1.30 | 0.94 | 1.57 | 1.75 | 1.25 | 1.49 | 1.52 | 1.57 | 1.40 | 1.34 | 1.46 |

### Example 2

### Exemplary High Dosage Mucoadhesive MTZ Aqueous-Based Gels

A number of exemplary high dosage mucoadhesive MTZ aqueous-based gels containing different concentrations of MTZ and utilizing a variety of different solvent systems were prepared and subject to a variety of different homogeneity, rheological, stability, biological and clinical tests as described in later examples. All MG-series and F-series gels were prepared using a similar process, described below. V-series gels were prepared by a slightly different process, also described below.

Preparation of MG and F-Series Gels.

Required amounts of methyl paraben and propyl paraben were weighed into a Duran® bottle followed by the aqueous phase (phosphate buffer or deionized water). The contents were thoroughly mixed to dissolve the preservatives. The remaining solvents (e.g., benzyl alcohol, ethanol, propylene glycol and PEG 400) were weighed into a separate Duran® bottle and mixed thoroughly followed by the required amount of metronidazole. The Duran® bottle was then placed in a water bath at 55° C. and stirred continuously until the metronidazole dissolved. The Duran® bottle was removed from the water bath, placed at room temperature and mixing was continued until the solution equilibrated to room temperature. The preservative solution was then added to the MTZ solution under constant stirring and the pH adjusted, if necessary. The required amount of gelling agent was weighed into a weighing boat and added to the above solution under constant stirring and stirred until the gelling agent fully hydrated. Care was taken to mix the contents thoroughly to ensure homogeneity.

For gels utilizing pH-sensitive or dependent gelling polymer, the pH of the MTZ solution can be adjusted to value within the optimal range for gelling prior to adding the gelling polymer, and the pH of the gel adjusted as need thereafter.

Preparation of V-Series Gels.

Required amounts of propylparaben, methylparaben and benzyl alcohol or phenoxyethanol were weighed into a vial and the contents stirred at ambient temperature with a magnetic stirrer until dissolved. An aliquot of this solution was transferred to a separate vial and the remaining ingredients, with the exception of the MTZ, water and carbopol were weighed into the vial and mixed at ambient temperature until dissolved. The MTZ was then weighed directly into this mixture and the mixture stirred at ambient temperature until the MTZ was dissolved. Water was then added, with stirring until a homogeneous solution was achieved, followed by the carbopol. The mixture was stirred overnight at ambient temperature with a magnetic stirrer set at a speed sufficient to create a vortex.

Preparation of Placebo Gels.

In several Experiments discussed herein, placebo gels are used as comparators or as controls. Placebo gels were prepared in a manner analogous to the corresponding active gel. For the placebos, the MTZ was omitted and replaced with an equivalent wt % of water or buffer, depending upon the aqueous phase used for the active gel.

Exemplary gels containing 1.18 wt % MTZ, 1.3 wt % MTZ, 1.5 wt % MTZ and 2.0 wt % MTZ are provided in Tables 3-6, below.

TABLE 3A
Exemplary Gels Containing 1.5 wt % MTZ

| | Composition (% w/w) | | | | | | | | |
|---|---|---|---|---|---|---|---|---|---|
| | MG03 | MG04 | MG08 | MG09 | MG26 | F17_{pH 6}* | F17_{pH 8} | F20_{pH 6} | F20_{pH 8} |
| Metronidazole | 1.50 | 1.50 | 1.50 | 1.50 | 1.50 | 1.50 | 1.50 | 1.50 | 1.50 |
| 1β-Hydroxypropylcyclodextrin | 20.00 | — | — | — | — | — | — | — | — |
| Purified Water | 76.70 | 51.70 | 49.45 | 48.45 | 42.00 | — | — | — | — |
| Phosphate buffer, pH 6 | — | — | — | — | — | 37.90 | — | 36.10 | — |
| Phosphate buffer, pH 8 | — | — | — | — | — | — | 37.90 | — | 36.10 |
| HEC (HHX) | 1.80 | 1.80 | — | — | — | — | — | — | — |
| Polycarbophil AA-1 | — | — | 3.00 | 5.00 | — | — | — | — | — |
| Carbomer 974P | — | — | 1.00 | — | 1.00 | 2.00 | 2.00 | 2.00 | 2.00 |
| EDTA, disodium salt | — | — | 0.05 | 0.05 | — | — | — | — | — |
| Methyl Paraben | — | — | — | — | — | 0.08 | 0.08 | 0.08 | 0.08 |
| Propyl Paraben | — | — | — | — | — | 0.02 | 0.02 | 0.02 | 0.02 |
| Octyldodecanol | — | — | — | — | 10.00 | — | — | — | — |
| Benzyl alcohol | — | 5.00 | 5.00 | 5.00 | 5.00 | 1.50 | 1.50 | 1.30 | 1.30 |
| Propylene glycol | — | 20.00 | 20.00 | 20.00 | 20.00 | 20.00 | 20.00 | 20.00 | 20.00 |
| PEG 400 | — | 20.00 | 20.00 | 20.00 | 20.00 | 37.00 | 37.00 | 39.00 | 39.00 |

*For gels F17 & F20, the pH designated is the pH of the phosphate buffer used to prepare the exemplary gel, and not the actual pH of of the prepared gel.

TABLE 3B
Exemplary Gels Containing 1.5 wt % MTZ

| | V1 | V2 | V3 | V4 | V5 | V6 | V7 | V8 |
|---|---|---|---|---|---|---|---|---|
| Metronidazole | 1.50 | 1.50 | 1.50 | 1.50 | 1.50 | 1.50 | 1.50 | 1.50 |
| Water | 37.90 | 37.90 | 37.90 | 37.90 | 37.90 | 37.90 | 37.90 | 37.90 |
| Carbomer 974P | 2.00 | 2.00 | 2.00 | 2.00 | 2.00 | 2.00 | 2.00 | 2.00 |
| Methyl paraben | 0.08 | 0.08 | 0.08 | 0.08 | 0.08 | 0.08 | 0.08 | 0.08 |
| Propyl paraben | 0.02 | 0.02 | 0.02 | 0.02 | 0.02 | 0.02 | 0.02 | 0.02 |
| Benzyl alcohol | 1.50 | 1.50 | 1.50 | 1.50 | 1.50 | 1.50 | 1.50 | 1.50 |
| Phenoxy | — | — | — | — | — | — | — | — |
| ethanol | | | | | | | | |
| Propylene | — | 13.00 | — | 20.00 | — | 20.00 | — | 13.00 |
| glycol | | | | | | | | |
| Propylene | 5.00 | 5.00 | 5.00 | 5.00 | 5.00 | 5.00 | — | — |
| carbonate | | | | | | | | |
| Dipropylene | — | — | 5.00 | 5.00 | — | — | 5.00 | 5.00 |
| glycol | | | | | | | | |
| Arlosolve | — | — | 15.00 | 15.00 | 15.00 | 15.00 | — | — |
| PEG 400 | 13.00 | — | 32.00 | 12.00 | 37.00 | 17.00 | 13.00 | — |
| PEG 200 | 39.00 | 39.00 | — | — | — | — | 39.00 | 39.00 |

TABLE 3C
Exemplary Gels Containing 1.5 wt % MTZ

| | V9 | V10 | V11 | V12 | V13 | V14 | V15 | V16 | V17 |
|---|---|---|---|---|---|---|---|---|---|
| Metronidazole | 1.50 | 1.50 | 1.50 | 1.50 | 1.50 | 1.50 | 1.50 | 1.50 | 1.50 |
| Water | 37.90 | 37.90 | 37.90 | 37.90 | 37.90 | 37.90 | 37.90 | 37.90 | 37.90 |
| Carbomer 974P | 2.00 | 2.00 | 2.00 | 2.00 | 2.00 | 2.00 | 2.00 | 2.00 | 2.00 |
| Methyl paraben | 0.08 | 0.08 | 0.08 | 0.08 | 0.08 | 0.08 | 0.08 | 0.08 | 0.08 |
| Propyl paraben | 0.02 | 0.02 | 0.02 | 0.02 | 0.02 | 0.02 | 0.02 | 0.02 | 0.02 |
| Benzyl alcohol | 1.50 | 1.50 | 1.50 | 1.50 | 1.50 | 1.50 | 1.50 | 1.50 | — |
| Phenoxy ethanol | — | — | — | — | — | — | — | — | 1.50 |
| Propylene glycol | 20.00 | 20.00 | — | 20.00 | — | 18.00 | 5.00 | 20.00 | 20.00 |
| Propylene carbonate | 2.00 | — | — | — | — | — | — | — | — |
| Dipropylene glycol | — | — | 5.00 | 5.00 | — | — | — | — | — |
| Arlosolve | 15.00 | — | 15.00 | 15.00 | — | — | 15.00 | 15.00 | — |
| PEG 400 | — | 17.00 | 37.00 | 17.00 | 18.00 | — | 37.00 | 22.00 | 37.00 |
| PEG 200 | 20.00 | 20.00 | — | — | 39.00 | 39.00 | — | — | — |

TABLE 4
Exemplary Gels Containing 2 wt % MTZ

| | Composition (% w/w) | | | |
|---|---|---|---|---|
| | MG18 | MG21 | MG23 | MG24 |
| Metronidazole | 2.00 | 2.00 | 2.00 | 2.00 |
| Purified Water | 39.95 | 51.00 | 48.95 | 41.45 |
| HEC (HHX) | — | 1.00 | — | — |
| Polycarbophil AA-1 | 3.00 | — | 3.00 | 1.50 |
| EDTA, disodium salt | 0.05 | — | 0.05 | 0.05 |
| Lutrol ® F127 | 10.00 | — | — | 10.00 |
| Tween ®60 | — | 1.00 | 1.00 | — |
| Benzyl alcohol | 5.00 | 5.00 | 5.00 | 5.00 |
| Propylene glycol | 20.00 | 20.00 | 20.00 | 20.00 |
| Polyethylene glycol 400 | 20.00 | 20.00 | 20.00 | 20.00 |

TABLE 5
Exemplary Gels Containing 1.3 wt % MTZ

| | Composition (% w/w) | | | | | | | | | | | |
|---|---|---|---|---|---|---|---|---|---|---|---|---|
| | MG32 | MG32PB | MG33 | MG33PB | MG34 | MG34PB | MG35 | MG35PB | MG36 | MG36PB | MG37 | MG37PB |
| Metronidazole | 1.30 | 1.30 | 1.30 | 1.30 | 1.30 | 1.30 | 1.30 | 1.30 | 1.30 | 1.30 | 1.30 | 1.30 |
| Purified Water | — | — | 54.70 | 54.60 | 54.70 | 54.60 | — | — | 55.20 | 55.10 | 55.20 | 55.10 |
| Phosphate buffer | 54.90 | 54.80 | — | — | — | — | 55.40 | 55.30 | — | — | — | — |
| pH 4 | | | | | | | | | | | | |
| HEC (HHX) | 1.80 | 1.80 | — | — | — | — | 1.80 | 1.80 | — | — | — | — |
| Polycarbophil AA-1 | — | — | 2.00 | 2.00 | — | — | — | — | 2.00 | 2.00 | — | — |
| Carbomer 974P | — | — | — | — | 2.00 | 2.00 | — | — | — | — | 2.00 | 2.00 |
| Methyl paraben | — | 0.08 | — | 0.08 | — | 0.08 | — | 0.08 | — | 0.08 | — | 0.08 |
| Propyl paraben | — | 0.02 | — | 0.02 | — | 0.02 | — | 0.02 | — | 0.02 | — | 0.02 |
| Benzyl alcohol | 2.00 | 2.00 | 2.00 | 2.00 | 2.00 | 2.00 | 1.50 | 1.50 | 1.50 | 1.50 | 1.50 | 1.50 |
| Propylene glycol | 15.00 | 15.00 | 15.00 | 15.00 | 15.00 | 15.00 | 20.00 | 20.00 | 20.00 | 20.00 | 20.00 | 20.00 |
| Polyethylene glycol | 25.00 | 25.00 | 25.00 | 25.00 | 25.00 | 25.00 | 20.00 | 20.00 | 20.00 | 20.00 | 20.00 | 20.00 |
| 400 | | | | | | | | | | | | |

TABLE 6
Exemplar Gels Containing 1.18 wt % MTZ

| | Composition (% w/w) | | |
|---|---|---|---|
| | MG42 | MG43 | MG44 |
| Metronidazole | 1.18 | 1.18 | 1.18 |
| Purified Water | 41.82 | 41.72 | 42.32 |
| HEC (HHX) | — | — | 0.90 |
| Polycarbophil AA-1 | 1.40 | — | 0.70 |
| Carbomer 974P | — | 1.50 | — |
| Lutrol ® F127 | 10.90 | 10.90 | 10.70 |
| Benzyl alcohol | 1.10 | 1.10 | 1.10 |
| Propylene glycol | 21.80 | 21.80 | 21.50 |
| Polyethylene glycol 400 | 21.80 | 21.80 | 21.60 |

### Example 3

### Shelf Stability of the Exemplary Gels

Various different embodiments of exemplary high dosage mucoadhesive MTZ aqueous-based gels were tested for long-term stability at 25° C. and 40° C.

For the study, 20 g of each test gel was stored in a sealed vial at 25±2° C./60±5% relative humidity (RH) and 40±2° C./75±5% RH. Back-up samples were also stored at −20° C., 2-8° C. and 30° C./65% RH for analysis upon failure of either real or accelerated time samples. Corresponding placebo gels without MTZ were stored along with the test gels.

Gels were removed from the storage cabinets after 1, 2, 3, 6 months, and for exemplary MG series test gels, also at 12 and 18 months, and tested for physical and chemical stability using microscopic analysis and viscosity analysis, as described below.

Microscopic Analysis.

Gels were viewed under a light microscope (Leica® DME). A small sample of the gel was placed onto a microscope slide using a micro-spatula. The microscope slide was covered with a cover glass and the gel viewed using the 40× objective. Active gels were compared to placebo gels for the presence of crystals.

If crystals were observed, the particle size was measured using the scale on a calibrated graticule (Olympus® Objective Micrometer, 0.01 mm). The microscope was set up so that the camera (Nikon Cool Pix® 4500 digital camera) was attached to the relay lens of the microscope and the 40× objective lens was set into place to view the sample. Camera settings: Image size: 1280×960 pixels, Image quality: Fine. Once a clear distinct view was obtained, pictures were taken (×400 magnification).

Viscosity Analysis.

The rheology of the MG series gels was measured at each time point (t=0, 1, 2, 3, 6, 12 and 18 months). A cone and plate measurement system is preferable for flow curve measurements as the shear rate is constant across the diameter of the truncated cone. Accordingly, a controlled shear rate ramp method was employed. A point on the flow curves at a shear rate of 0.16 s⁻¹ was used to compare each gel and the viscosity determined. A Bohlin CVO 100 rheometer and the rheometer settings used are in Table 7 below:

TABLE 7
Rheometer settings for the controlled shear rate ramp

| Measurement type/diameter | Cone and plate/4°/40 mm |
|---|---|
| Gap size | 150 μm |
| Shear rate range | From 0.001-1.0 s−1 |
| Duration shear rate | 180 s |
| Progression of shear rate | Linear |
| increments | |
| Temperature of sample | 25° C. ± 0.1° C. |
| Isothermal conditions | Measurements forced to wait until sample |
| | at temperature, time out after 900 s |
| Thermal equilibration time | 0 s once temperature was 25° C. ± 0.1 |
| | before first measurements were made. |

The viscosity of the F series test gels was tested by using the same method.

Gels were also tested for MTZ content, benzyl alcohol content, paraben content and pH. For the extraction, 0.5 g±20 mg of gel was weighed into a 25 ml volumetric flask and diluted with approximately 20 ml of sample diluient (3:7 methanol/10 mM KH₂PO₄). The mixture was vortexed until the gel dispersed into solution (approximately 5 min). The solution was brought to volume with sample diluents and vortex mixed for approximately 1 minute. Approximately 14 ml of the sample was centrifuged at 3500 rpm for 10 min., the resultant supernatant filtered through a 0.45 μm PTFE syringe filter, discarding the first 2 ml. The remaining filtrate was analyzed by HPLC Method 2 (parameters in Table 8, below). In addition to MTZ, the recovery of benzyl alcohol, methyl paraben and propyl paraben was also determined.

TABLE 8
HPLC Method 2

| HPLC System | Waters 2695 Alliance HPLC System | | |
|---|---|---|---|
| | Waters 2996 Photo-Diode Array Detector | | |
| | Waters Empower Data Processing Software | | |
| | (version 6.10.01.00) | | |
| Column | Phenomenex luna C18(2) 5 μm, 250 × 4.6 mm | | |
| | lot number 479 458-61 | | |
| Guard Column | Phenomenex Security guard C18, 4 × 3 mm | | |
| Detection | 254 nm | | |
| Sample Temperature | 25 ± 2° C. | | |
| Column Temperature | 25 ± 2° C. | | |
| Flow Rate | 1 mL/min | | |
| Mobile Phase | Mobile phase A: 0.01M potassium dihydrogen | | |
| | phosphate dihydrate | | |
| | Mobile phase B: 100% methanol | | |
| | Time | Mobile | Mobile |
| | (min) | phase A (%) | phase B (%) |
| Gradient | 0 | 80 | 20 |
| | 10 | 80 | 20 |
| | 18 | 28 | 72 |
| | 22 | 28 | 72 |
| | 23 | 80 | 20 |
| | 28 | 80 | 20 |

| | |
|---|---|
| Injection Volume | 10 μl |
| Run Time | 28 min |
| MTZ Retention Time | 9.0 min |
| Needle Wash | 60:40 methanol (HPLC grade):deionized water |
| | (MilliQ 18.2 MΩ) |
| Pump Wash | 60:40 methanol (HPLC grade):deionized water |
| | (MilliQ 18.2 MΩ) |

Results. MTZ Recovery.

Table 9 shows the % recovery of MTZ from the MG series test gels. The percentage recovery of MTZ from the MG-series test gels stored at 25° C. and 40° C. for 6 months is comparable to t=0 data. The % peak purity for all the gels is found to be 100%, indicating that MTZ is stable in all the MG-series gels tested. The percentage recovery of MTZ from MG33PB and MG32PB stored at 25° C. and 40° C. for 18 months is comparable to t=0 data.

TABLE 9
Stability of Exemplary Gels at 25° C. and 40° C.
Data show % recovery of MTZ from the gel (n = 3, mean ± SEM)

| T (months) | MG32PB | MG33PB | MG34PB | MG35PB | MG36PB | MG37PB |
|---|---|---|---|---|---|---|
| t = 0* | 102.68 ± 0.25 | 102.68 ± 0.26 | 102.07 ± 0.21 | 102.15 ± 0.16 | 101.97 ± 0.53 | 102.68 ± 0.15 |
| t = 1 m 25° C. | 102.19 ± 0.16 | 102.12 ± 0.06 | 101.91 ± 0.23 | 103.13 ± 0.38 | 101.91 ± 0.15 | 101.95 ± 0.09 |
| t = 1 m 40° C. | 102.48 ± 0.06 | 101.38 ± 0.18 | 101.50 ± 0.13 | 104.12 ± 0.37 | 101.26 ± 0.20 | 101.85 ± 0.22 |
| t = 2 m 25° C. | 101.90 ± 0.11 | 101.55 ± 0.11 | 101.98 ± 0.13 | 102.29 ± 0.18 | 101.82 ± 0.13 | 102.43 ± 0.20 |
| t = 2 m 40° C. | 101.65 ± 0.35 | 99.59 ± 0.85 | 99.82 ± 0.60 | 101.60 ± 0.20 | 100.61 ± 0.56 | 100.64 ± 0.52 |
| t = 3 m 25° C. | 100.87 ± 0.31 | 100.44 ± 0.04 | 100.83 ± 0.33 | 102.20 ± 0.41 | 101.36 ± 0.04 | 102.02 ± 0.10 |
| t = 3 m 40° C. | 100.70 ± 0.11 | 98.96 ± 0.79 | 98.09 ± 0.44 | 99.71 ± 0.07 | 97.36 ± 0.18 | 98.55 ± 0.05 |
| t = 6 m 25° C. | 101.68 ± 0.54 | 100.87 ± 0.50 | 101.68 ± 0.25 | 101.26 ± 0.11 | 100.77 ± 0.29 | 102.57 ± 0.11 |
| t = 6 m 40° C. | 100.97 ± 0.67 | 101.13 ± 0.42 | 99.87 ± 0.30 | 101.08 ± 0.35 | 99.40 ± 0.37 | 100.46 ± 0.22 |
| t = 12 m 25° C. | n/a | 101.15 ± 0.23 | n/a | n/a | n/a | n/a |
| t = 12 m 40° C. | n/a | 98.25 ± 0.07 | n/a | n/a | n/a | n/a |
| t = 18 m 25° C. | 103.53 ± 2.42 | 99.91 ± 0.77 | n/a | n/a | n/a | n/a |
| t = 18 m 40° C. | 99.85 ± 0.28 | 98.03 ± 1.69 | n/a | n/a | n/a | n/a |

*% recovery at t = 0 was performed n = 6 ± SEM for homogeneity

MTZ recovery and purity data from the F-series test gels are provided in Tables 10 and 11, below.

TABLE 10
MTZ Recovery following storage of formulations at 25°
C. and 40° C. over a 6 month period.

| | Metronidazole assay (% recovery) | | | | |
|---|---|---|---|---|---|
| Formu- | | t = 4 weeks | | t = 6 months | |
| lations | t = 0 | 25° C. | 40° C. | 25° C. | 40° C. |
| F17_{pH 6} | 98.88 ± 0.15 | 99.09 ± 0.93 | 100.39 ± 1.14 | 99.65* | 98.93* |
| Active | | | | | |
| F17_{pH 8} | 99.51 ± 0.65 | 97.87 ± 4.27 | 99.55 ± 0.67 | 99.95* | 95.63* |
| Active | | | | | |
| F20_{pH 6} | 99.10 ± 0.54 | 99.09 ± 0.93 | 100.39 ± 1.14 | 100.01* | 98.32* |
| Active | | | | | |
| F20_{pH 8} | 99.37 ± 0.17 | 97.87 ± 4.27 | 99.55 ± 0.67 | 100.76* | 99.11* |
| Active | | | | | |

*Due to the limited amount of formulation available at the t = 6 month time point only n = 1 extraction was performed for both the recovery and peak purity.

TABLE 11
MTZ Peak Purity following storage of formulations at 25°
C. and 40° C. over a 6 month period.

| | Metronidazole assay (% recovery) | | | | |
|---|---|---|---|---|---|
| | | t = 4 weeks | | t = 6 months | |
| Formulations | t = 0 | 25° C. | 40° C. | 25° C. | 40° C. |
| F17_{pH 6} Active | 100.00 ± 0.00 | 100.00 ± 0.00 | 100.00 ± 0.00 | 100.00* | 100.00* |
| F17_{pH 8} Active | 100.00 ± 0.00 | 100.00 ± 0.00 | 100.00 ± 0.00 | 100.00* | 100.00* |
| F20_{pH 6} Active | 100.00 ± 0.00 | 100.00 ± 0.00 | 100.00 ± 0.00 | 100.00* | 100.00* |
| F20_{pH 8} Active | 100.00 ± 0.00 | 100.00 ± 0.00 | 100.00 ± 0.00 | 100.00* | 100.00* |

*n = 1 only.

The data showed that no change in metronidazole recovery was observed after t=6 months storage and furthermore, no additional peaks (100% peak purity) were present in any of the formulations tested. As such the data showed that there appeared to be no stability issues of metronidazole in the formulations tested over the duration of the study.

Results. pH.

The pHs of the MG-series test gels are provided in Table 12, below. The pHs of the F-series test gels are provided in Table 13, below.

TABLE 12
pH of Exemplary Gels Stored at 25° C. and 40° C.

| | | t = 1 m | | t = 2 m | | t = 3 m | | t = 6 m | | t = 12 m | | t = 18 m | |
|---|---|---|---|---|---|---|---|---|---|---|---|---|---|
| | t = 0 | 25° C. | 40° C. | 25° C. | 40° C. | 25° C. | 40° C. | 25° C. | 40° C. | 25° C. | 40° C. | 25° C. | 40° C. |
| Formulation | | | | | | | | | | | | | |
| (Placebos) | | | | | | | | | | | | | |
| MG32PB | 5.1 | 4.9 | 5.2 | 5.2 | 5.0 | 5.0 | 4.9 | 5.1 | 4.8 | n/a | n/a | 5.2 | 5.1 |
| MG33PB | 3.9 | 3.8 | 3.9 | 3.9 | 3.8 | 3.7 | 3.7 | 3.8 | 3.8 | 4.1 | 3.8 | 3.9 | 3.9 |
| MG34PB | 4.0 | 3.8 | 3.9 | 4.0 | 3.9 | 3.7 | 3.8 | 3.8 | 3.9 | n/a | n/a | n/a | n/a |
| MG35PB | 5.1 | 5.1 | 5.0 | 5.2 | 4.9 | 5.1 | 4.9 | 5.0 | 4.9 | n/a | n/a | n/a | n/a |
| MG36PB | 3.8 | 3.7 | 3.7 | 3.9 | 3.7 | 3.5 | 3.5 | 3.6 | 3.6 | n/a | n/a | n/a | n/a |
| MG37PB | 3.8 | 3.7 | 3.6 | 3.6 | 3.8 | 3.5 | 3.5 | 3.6 | 3.6 | n/a | n/a | n/a | n/a |
| Formulation | | | | | | | | | | | | | |
| (Actives) | | | | | | | | | | | | | |
| MG32PB | 5.1 | 5.1 | 5.1 | 5.2 | 5.1 | 5.1 | 5.1 | 5.1 | 5.0 | n/a | n/a | 5.2 | 5.1 |
| MG33PB | 3.9 | 4.0 | 3.9 | 4.2 | 3.9 | 3.8 | 4.0 | 4.1 | 4.0 | 4.2 | 4.3 | 5.0 | 4.4 |
| MG34PB | 4.0 | 4.1 | 4.0 | 4.1 | 4.1 | 3.7 | 3.9 | 3.9 | 4.0 | n/a | n/a | n/a | n/a |
| MG35PB | 5.2 | 4.9 | 5.1 | 5.3 | 5.2 | 5.2 | 5.1 | 5.1 | 5.1 | n/a | n/a | n/a | n/a |
| MG36PB | 4.0 | 3.9 | 3.9 | 4.1 | 4.0 | 3.7 | 3.7 | 4.0 | 4.1 | n/a | n/a | n/a | n/a |
| MG37PB | 4.0 | 3.9 | 4.0 | 4.1 | 3.9 | 3.8 | 3.9 | 4.1 | 4.0 | n/a | n/a | n/a | n/a |

TABLE 13
pH of Exemplary Gels following storage at 25°
C. and 40° C. over a 6 month period.

| | pH | | | | |
|---|---|---|---|---|---|
| | | t = 4 weeks | | t = 6 months | |
| Formulations | t = 0 | 25° C. | 40° C. | 25° C. | 40° C. |
| F17_{pH 6} Active | 5.32 | 5.36 | 5.33 | 5.31 | 5.36 |
| F17_{pH 6} Placebo | 5.50 | 5.49 | 5.52 | 5.38 | 5.55 |
| F17_{pH 8} Active | 5.41 | 5.43 | 5.37 | 5.40 | 5.38 |
| F17_{pH 8} Placebo | 5.38 | 5.37 | 5.41 | 5.40 | 5.40 |
| F20_{pH 6} Active | 5.41 | 5.39 | 5.43 | 5.45 | 5.40 |
| F20_{pH 6} Placebo | 5.37 | 5.42 | 5.41 | 5.39 | 5.40 |
| F20_{pH 8} Active | 5.43 | 5.49 | 5.52 | 5.51 | 5.51 |
| F20_{pH 8} Placebo | 5.42 | 5.48 | 5.46 | 5.38 | 5.41 |

Results. Viscosity.

The viscosities of the F-series test gels are provided in Table 14, below. The viscosities of the MG-series test gels are provided in Table 15, below.

TABLE 14
Viscosities of Exemplary Gels

| | Bohlin viscosity (cPs) | | | | |
|---|---|---|---|---|---|
| | | T = 1 month | | T = 6 months | |
| Formulations | T = 0 | 25° C. | 40° C. | 25° C. | 40° C. |
| F17_{pH 6} Active | 159898 | 149357 | 173283 | 198329 | 178628 |
| F17_{pH 6} Placebo | 125558 | 127804 | 130269 | 129699 | 137295 |
| F17_{pH 8} Active | 452384 | 440864 | 326494 | 280644 | 285431 |
| F17_{pH 8} Placebo | 372158 | 373645 | 375461 | 355290 | 297754 |
| F20_{pH 6} Active | 176865 | 185300 | 200967 | 186238 | 205114 |
| F20_{pH 6} Placebo | 186546 | 188489 | 167475 | 174956 | 172244 |
| F20_{pH 8} Active | 425577 | 341850 | 347529 | 292167 | 262989 |
| F20_{pH 8} Placebo | 390095 | 369209 | 363335 | 361390 | 314570 |

TABLE 15
Viscosity of Exemplary Gels Stored at 25° C. and 40° C.

| Time | MG32PB | | MG33PB | | MG34PB | | MG35PB | | MG36PB | | MG37PB | |
|---|---|---|---|---|---|---|---|---|---|---|---|---|
| (months) | Placebo | Active | Placebo | Active | Placebo | Active | Placebo | Active | Placebo | Active | Placebo | Active |
| | Viscosity (mPas) at 25° C. | | | | | | | | | | | |
| t = 0 | 299907 | 285766 | 263415 | 316624 | 333411 | 415236 | 287357 | 292432 | 308055 | 291714 | 324138 | 371399 |
| t = 1 | 309065 | 325695 | 270402 | 294917 | 334730 | 375060 | 290940 | 300019 | 271073 | 274503 | 251456 | 339551 |
| t = 2 | 331546 | 263264 | 273861 | 287725 | 333193 | 376395 | 291295 | 268178 | 267250 | 276140 | 231617 | 359769 |
| t = 3 | 311637 | 294178 | 259886 | 276503 | 315363 | 372616 | 305701 | 297965 | 243066 | 258491 | 212763 | 336410 |
| t = 6 | 258433 | 250116 | 261494 | 308102 | 328419 | 374120 | 242216 | 298475 | 247567 | 275626 | 219261 | 337051 |
| t = 12 | n/a | n/a | 215339 | 235108 | n/a | n/a | n/a | n/a | n/a | n/a | n/a | n/a |
| t = 18 | 275223 | 293346 | 194215 | 219741 | n/a | n/a | n/a | n/a | n/a | n/a | n/a | n/a |
| | Viscosity (mPas) at 40° C. | | | | | | | | | | | |
| t = 0 | 299907 | 285766 | 263415 | 316624 | 333411 | 415236 | 287357 | 292432 | 308055 | 291714 | 324138 | 371399 |
| t = 1 | 319774 | 280639 | 361158 | 263315 | 329030 | 360147 | 301443 | 280435 | 305288 | 261720 | 206307 | 335181 |
| t = 2 | 305906 | 242894 | 367321 | 259016 | 376333 | 356693 | 277249 | 243496 | 364213 | 258584 | 287505 | 334457 |
| t = 3 | 281581 | 276653 | 298214 | 253704 | 349430 | 316822 | 256484 | 280481 | 272173 | 219989 | 239640 | 307145 |
| t = 6 | 179966 | 228019 | 254421 | 241973 | 284824 | 315104 | 183874 | 216715 | 191400 | 231610 | 178624 | 280642 |
| t = 12 | n/a | n/a | 115088 | 187612 | n/a | n/a | n/a | n/a | n/a | n/a | n/a | n/a |
| t = 18 | 57878 | 199306 | 139706 | 198759 | n/a | n/a | n/a | n/a | n/a | n/a | n/a | n/a |

### Example 4

### Local Delivery and Skin Penetration Properties of the Exemplary Gels

The in vitro skin permeation properties of several exemplary high dosage MTZ aqueous-based gels were tested in a Franz cell with a full thickness of human abdominoplasty skin obtained with informed consent. For the studies, all subcutaneous fat was removed with a scalpel and the skin was mounted in-between the donor and receiver compartments.

For the experiment, ¹⁴C-labeled MTZ MG-series test gels were prepared as described in Example 2 with the quantities of ingredients listed in Table 16, below. In Table 16, all quantities are in mg, which the exception of ¹⁴C-labeled MTZ, which is in μl.

The ¹⁴C-labeled MTZ (GE Healthcare) had a specific activity of 57 mCi/mmol with a radiochemical purity of 99.2%. ¹⁴C-labeled MTZ stock solution was prepared by adding 3.5 ml water to 37 MBq of ¹⁴C-labeled MTZ and vortex mixing until the ¹⁴C-labeled MTZ dissolved.

As a control for the MG-series test gels, 10 g of a 0.75 wt % ¹⁴C-labeled MTZ gel corresponding to FDA approved 0.75 wt % MTZ gels (such as, for example, METROGEL VAGINAL®) was prepared as follows:

Paraben Phase:

Methyl paraben (8.03 mg), propylparaben (2.05 mg) and propylene glycol (303.28 mg) were weighed into a 28 ml glass vial and stirred until dissolved. The vial was then equilibrated at 55° C. and water (3.5 g) added while stirring. MTZ (75 mg) was added and stirring continued until all the drug had dissolved. ¹⁴C-labeled MTZ stock solution (465 μl) was then added while stirring.

Carbomer Phase:

Disodium EDTA (5.03 mg) was weighed into a 28 ml glass vial to which purified water (5.698 g) was added. A magnetic flea was used to stir the formulation until all the disodium EDTA was fully dissolved. Carbomer 974P (200.89 mg) was added and the formulation left stirring overnight to allow the polymer to hydrate.

Combination:

The paraben phase was added to the carbomer phase while stirring. The paraben phase was rinsed into the carbomer phase using water (200 mg). The gel was left overnight to cool to room temperature.

F-series test gels containing ¹⁴C-labeled MTZ were prepared by spiking the test gels with 465 μl ¹⁴C-labled MTZ stock. For the F-series test gels, a 0.75% MTZ control was prepared by spiking commercial 0.75% MTZ METROGEL with 465 μl ¹⁴C-labled MTZ stock.

TABLE 16
Exact Composition of Radio Labeled MTZ MG-Series Test Gels and 0.75% MTZ Control

| | MG32PB | MG33PB | MG34PB | MG35PB | MG36PB | MG37PB |
|---|---|---|---|---|---|---|
| Metronidazole | 130.57 | 130.40 | 129.38 | 129.79 | 129.49 | 131.07 |
| ¹⁴C-labeled MTZ (μl) | 465 | 465 | 465 | 465 | 465 | 465 |
| Purified Water | — | 4999.24 | 5007.60 | — | 5049.89 | 5059.36 |
| Phosphate buffer pH 4 | 5013.41 | — | — | 5066.56 | — | — |
| Hydroxyethylcellulose (HHX) | 181.25 | — | — | 180.54 | — | — |
| Polycarbophil AA-1 | — | 200.96 | — | — | 201.23 | — |
| Carbomer 974P | — | — | 205.17 | — | — | 202.52 |
| Methyl paraben | 8.00 | 8.05 | 8.05 | 7.99 | 8.12 | 8.08 |
| Propyl paraben | 2.05 | 2.00 | 2.18 | 2.06 | 2.09 | 1.96 |
| Benzyl alcohol | 208.29 | 207.47 | 203.66 | 158.42 | 144.64 | 143.21 |
| Propylene glycol | 1496.30 | 1502.17 | 1505.17 | 1994.41 | 2008.30 | 2007.37 |
| Polyethylene glycol 400 | 2497.40 | 2495.34 | 2501.84 | 2008.76 | 2018.83 | 2002.24 |
| Total (mg) | 10002.27 | 10010.63 | 10028.05 | 10013.53 | 10027.59 | 10020.81 |
| Result | Clear thick gel | Clear thick gel | Clear thick gel | Clear thick gel | Clear thick gel | Clear thick gel |

For the experiment, a full thickness human skin was mounted in a Franz cell with phosphate buffer (pH 4) as receiver fluid to ensure sink conditions. A finite dose of test gel equivalent to 10 mg/cm² was applied to the membrane and the diffusion of ¹⁴C-labeled MTZ determined over time.

The test gels (7 μl) were applied to the surface of the membrane using a positive displacement pipette. For the MG-series gels, due to the large number of gels tested, the study was split into several separate experiments. Two skin donors were randomly assigned across all experiments so that each gel was tested on both skin donors (n=6 cells per gel).

The receptor compartment of the Franz cells was filled with receiver fluid and the cells fixed in a water bath maintained at 37° C. The receptor chamber contents were continuously agitated by small magnetic followers. At t=1, 2, 3, 4, 6, 8 and 24 h, samples of receiver fluid were taken from the receptor compartment, and replaced with fresh receiver medium and assayed by scintillation counting.

At the end of the experiment, a mass balance was carried out, analysing donor chamber, surface residue, Stratum corneum (SC), remaining epidermis, dermis and receiver chamber. The method involved removal of the SC by tape stripping and processing of the remaining epidermal layer and dermis using standard procedures. The methods are described briefly below.

Unabsorbed Formulation:

the surface of each Franz cell donor chamber was wiped gently with a cotton bud using 5 clockwise and anti-clockwise movements. The procedure was repeated on four occasions using alternate wet (previously immersed in receiver fluid) and dry cotton buds. The cotton buds were added to scintillation cocktail before analysis. Two tape strips were removed from the skin and regarded as the unabsorbed formulation and included in the total surface activity. The tape strips were placed into a scintillation vial to which 1 mL of water was added. These were left to soak for 72 h to allow the formulation to dissolve and disperse into water. Scintillation cocktail (4 mL) was added to the vial prior to analysis by liquid scintillation (LSC). The surface of each Franz cell receiver chamber was wiped gently with a cotton bud using five clockwise and anti-clockwise movements. This procedure was repeated on two occasions using alternate wet (previously immersed in receiver fluid) and dry cotton buds. The cotton buds were added to scintillation cocktail before analysis.

Stratum Corneum (SC):

SC was removed by carefully tape stripping the skin ten times using Scotch adhesive tape. The first five tape strips were placed together in one scintillation vial and the second five together in a second vial. 1 mL of water was added to each vial and these were left to soak for 72 h to allow the formulation to dissolve and disperse into the water. Scintillation cocktail (4 mL) was added to the vial prior to analysis by LSC.

Epidermis:

The remaining section of the epidermis (following tape stripping) was carefully removed from the dermis with a scalpel. The epidermis was placed into a glass vial containing 2 ml of Soluene 350 and incubated at 50° C. for 72 h before analysis by LSC.

Dermis:

The remaining dermal layer was placed in to a glass vial containing 2 ml of Soluene 350 and digested by incubation at 50° C. for 72 h before analysis by LSC.

Results.

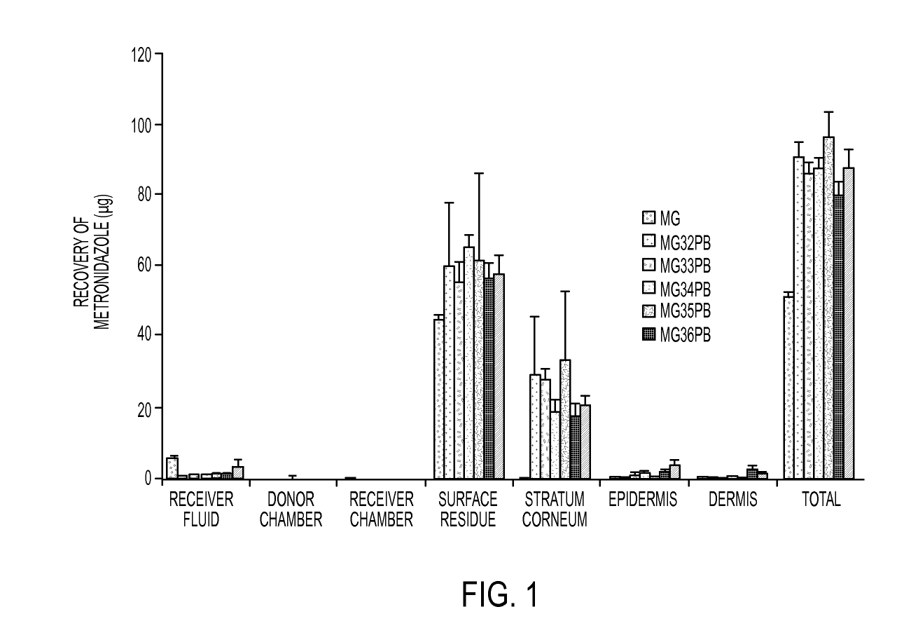

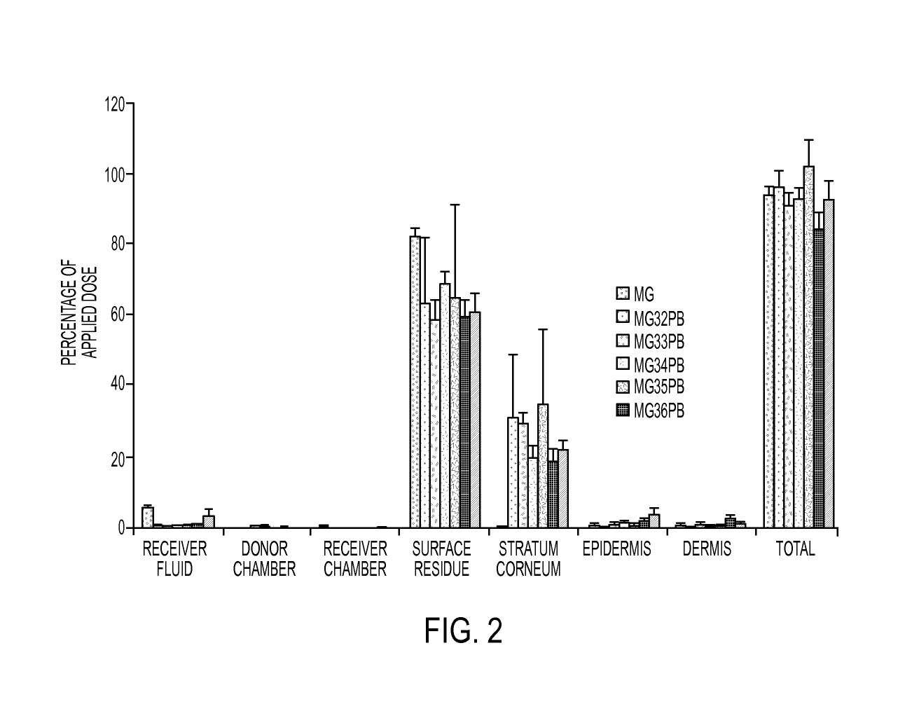

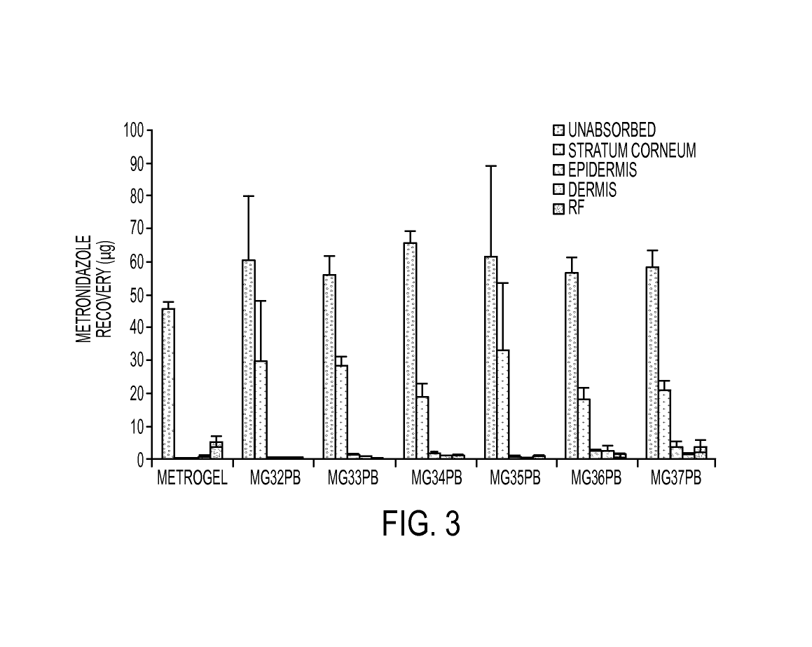

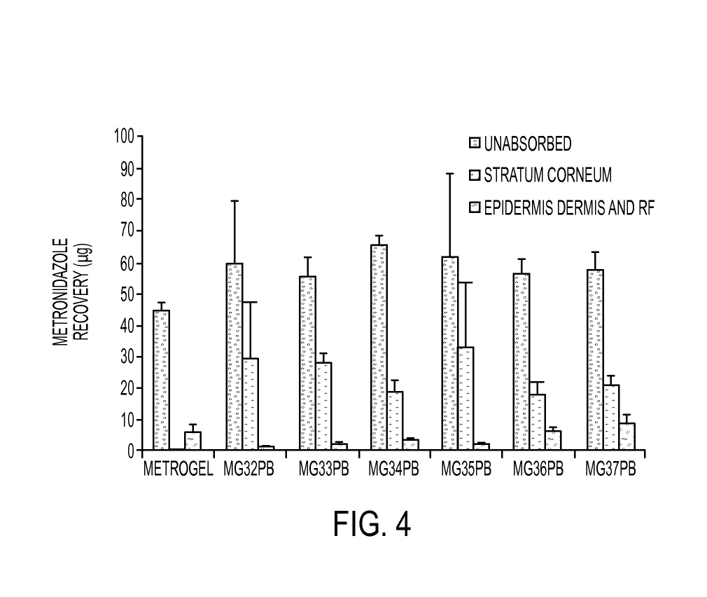

The flux of MTZ released from the F-series gels is provided in Table 17, below. The amounts of ¹⁴C-labeled MTZ recovered from the various skin layers for the MG-series test gels are provided in Table 18, below. These data are represented graphically in FIG. 1. The same data, represented as a percentage of the applied dose are provided in Table 19, below and graphically in FIG. 2. Additional graphical representations are provided in FIGS. 3 and 4.

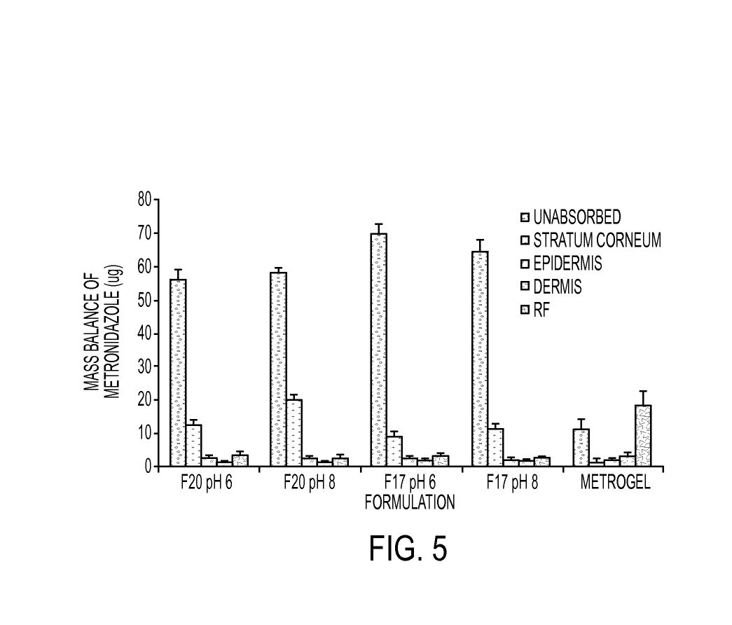

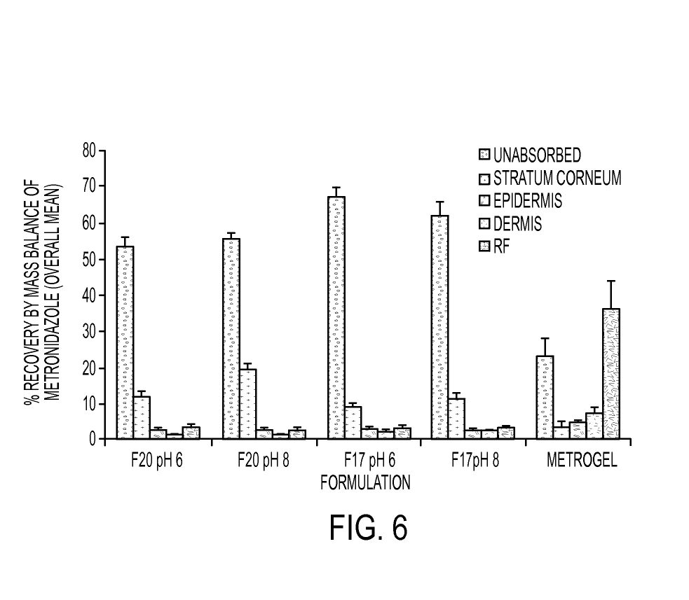

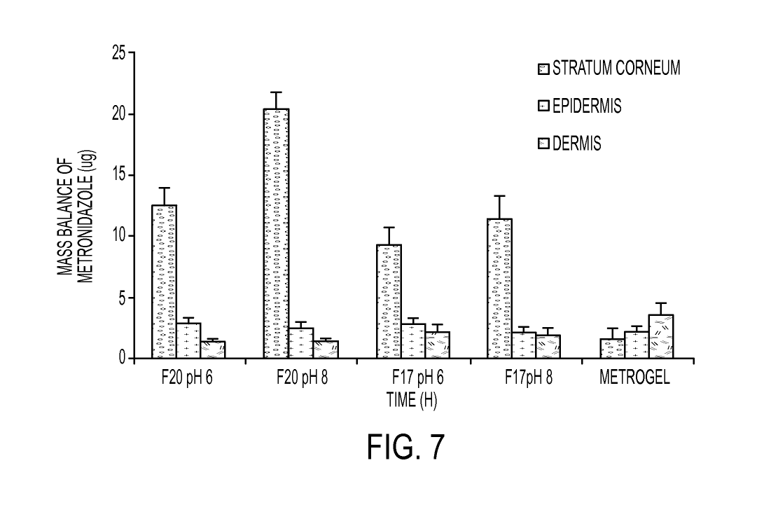

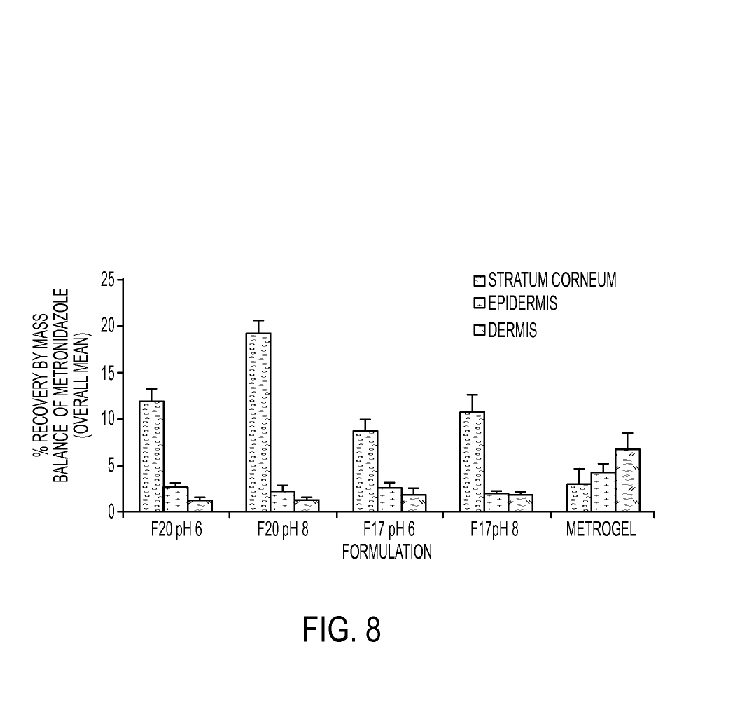

The mean amounts of ¹⁴C-labeled MTZ recovered in an experiment carried out with exemplary F-series gels are illustrated in FIG. 5. The same data represented as the percentages of total MTZ dose applied are illustrated in FIG. 6. The same data plotted at a different scale for related skin layers are illustrated in FIGS. 7 and 8.

TABLE 17
Mean flux of MTZ (μg cm−2 h−1) from 0
to 24 h (mean ± SD, n = 6)

| Formulations | Flux μg/cm2/h (0-24 h) |
|---|---|
| F17_{pH 6} (1.50% metronidazole) | 4.17 ± 0.28 |
| F17_{pH 8} (1.50% metronidazole) | 4.18 ± 0.67 |
| F20_{pH 6} (1.50% metronidazole) | 3.86 ± 0.46 |
| F20_{pH 8} (1.50% metronidazole) | 3.89 ± 0.53 |
| Metrogel (0.75% metronidazole) | 4.43 ± 0.37 |

TABLE 18
Mean (±SEM) Recovery (μg) of ¹⁴C MTZ from Gels Applied to Full Thickness Human Skin

| | | Receiver | Donor | Receiver | Surface | Stratum | | | |
|---|---|---|---|---|---|---|---|---|---|
| Test Gel | N | Fluid | Chamber | Chamber | Residue | corneum | Epidermis | Dermis | Total |
| Metrogel ® | 4 | 4.83 ± 0.97 | 0.01 ± 0.00 | 0.18 ± 0.06 | 45.03 ± 1.20 | 0.37 ± 0.74 | 0.47 ± 0.08 | 0.65 ± 0.17 | 51.54 ± 1.09 |
| MG32PB | 6 | 0.47 ± 0.10 | 0.01 ± 0.00 | 0.01 ± 0.00 | 59.60 ± 17.84 | 29.47 ± 16.56 | 0.54 ± 0.05 | 0.60 ± 0.07 | 91.00 ± 4.03 |
| MG33PB | 6 | 0.31 ± 0.27 | 0.10 ± 0.10 | 0.00 ± 0.00 | 55.69 ± 5.42 | 28.14 ± 2.66 | 1.17 ± 0.48 | 0.72 ± 0.14 | 86.01 ± 3.42 |
| MG34PB | 5 | 1.02 ± 0.11 | 0.35 ± 0.35 | 0.05 ± 0.02 | 65.23 ± 3.29 | 18.78 ± 3.51 | 1.69 ± 0.44 | 0.78 ± 0.18 | 87.77 ± 2.69 |
| MG35PB | 5 | 0.75 ± 0.16 | 0.02 ± 0.01 | 0.02 ± 0.00 | 61.42 ± 24.96 | 33.15 ± 19.86 | 0.61 ± 0.11 | 0.55 ± 0.04 | 96.34 ± 7.21 |
| MG36PB | 5 | 1.17 ± 0.25 | 0.02 ± 0.01 | 0.05 ± 0.00 | 56.47 ± 4.38 | 17.80 ± 3.45 | 2.09 ± 0.55 | 2.66 ± 1.04 | 79.97 ± 3.71 |
| MG37PB | 5 | 3.62 ± 2.02 | 0.03 ± 0.02 | 0.07 ± 0.04 | 57.89 ± 4.95 | 20.98 ± 2.56 | 3.79 ± 1.46 | 1.31 ± 0.58 | 86.47 ± 5.43 |

“N” is the number tested

TABLE 19
Mean Percentage Recovery (±SEM) of ¹⁴C MTZ from Gels Applied to Full Thickness Human Skin

| | | Receiver | | Receiver | | | | | |
|---|---|---|---|---|---|---|---|---|---|
| Gel | N | Fluid | Donor Chamber | Chamber | Surface Residue | Stratum corneum | Epidermis | Dermis | Total |
| Metrogel ® | 4 | 9.20 ± 1.85 | 0.01 ± 0.00 | 0.34 ± 0.11 | 85.78 ± 2.28 | 0.71 ± 0.14 | 0.90 ± 0.15 | 1.24 ± 0.33 | 98.17 ± 2.07 |
| MG32PB | 6 | 0.52 ± 0.11 | 0.01 ± 0.00 | 0.01 ± 0.00 | 65.89 ± 19.60 | 32.38 ± 18.20 | 0.59 ± 0.06 | 0.66 ± 0.08 | 100.00 ± 4.43 |
| MG33PB | 6 | 0.34 ± 0.03 | 0.11 ± 0.11 | 0.00 ± 0.00 | 61.20 ± 5.96 | 30.92 ± 2.92 | 1.29 ± 0.53 | 0.79 ± 0.15 | 94.52 ± 3.76 |
| MG34PB | 5 | 1.12 ± 0.12 | 0.39 ± 0.38 | 0.06 ± 0.02 | 71.68 ± 3.62 | 20.64 ± 3.86 | 1.86 ± 0.48 | 0.86 ± 0.20 | 96.45 ± 2.96 |
| MG35PB | 5 | 0.82 ± 0.18 | 0.02 ± 0.01 | 0.02 ± 0.00 | 67.49 ± 27.43 | 36.43 ± 21.82 | 0.67 ± 0.12 | 0.60 ± 0.04 | 105.87 ± 7.92 |
| MG36PB | 5 | 1.29 ± 0.27 | 0.02 ± 0.01 | 0.05 ± 0.00 | 62.06 ± 4.81 | 19.56 ± 3.79 | 2.30 ± 0.60 | 2.92 ± 1.14 | 87.88 ± 4.08 |
| MG37PB | 5 | 3.98 ± 2.22 | 0.03 ± 0.02 | 0.08 ± 0.04 | 63.62 ± 5.44 | 23.06 ± 2.81 | 4.17 ± 1.61 | 1.44 ± 0.64 | 95.02 ± 5.97 |

“N” is the number tested

There was no statistical difference (p>0.05) between the amount of MTZ detected in the donor chamber, receiver chamber, epidermis, and dermis for any of the formulations tested. However, significant differences in the amounts detected in the receiver fluid and the Stratum corneum were observed with the high dosage MTZ MG-series and F-series test gels as compared to 0.75% Metrogel®. The levels of MTZ observed in the receiver fluid following application of Metrogel® (4.83±0.97 μg) were 4-16 higher (2-9 fold when normalized for concentration; p<0.05) than from the MG-series test gels (min 0.31±0.27 μg from MG33PB and max 1.17±0.25 μg from MG36PB), with the possible exception of MG37PB (3.62±2.02 μg). However the levels of ¹⁴C-labeled MTZ in the Stratum corneum following application of the exemplary MG-series test gels (min 17.80±3.45 μg from MG36PB and max 33.15±19.86 μg from MG35PB) were 50-90 fold higher (29-53 fold higher when normalized to concentration) than those obtained for Metrogel® (0.37+0.74 μg). This difference was reflected in the amount of ¹⁴C-labeled MTZ recovered unabsorbed from the skin surface were the amount of drug recovered from the Metrogel® formulation (45.03±1.20 μg) was comparable to the exemplary test gels (min 55.69±5.42 μg from MG33PB and max 65.23±3.29 μg from MG34PB) despite the difference in initial concentrations. Similar results were observed with the F-series test gels. Thus, a greater quantity of MTZ is retained in the superficial layers of the skin in the experiment with the high dosage MTZ aqueous-based gels described herein as compared to conventional 0.75 wt % MTZ gels, while at the same time yielding lower levels in receiver fluid, corresponding to expected lower systemic levels when applied topically to skin.

### Example 5

### Skin Penetration with Unlabeled MTZ

A skin penetration experiment similar to that described in Example 4 was carried out with exemplary high dosage aqueous-based MTZ gels F17_{pH8}, V1, V2, V3, V4, V5, V6, V7, V8, V9, V10, V11, V12, V13, V14, V15, V16, and V17 and two commercial 0.75 wt % MTZ control gels, one from Galderma and one from Medicis. For this experiment, the MTZ was not radio labeled. MTZ recovered from the various skin layers was quantified by HPLC.

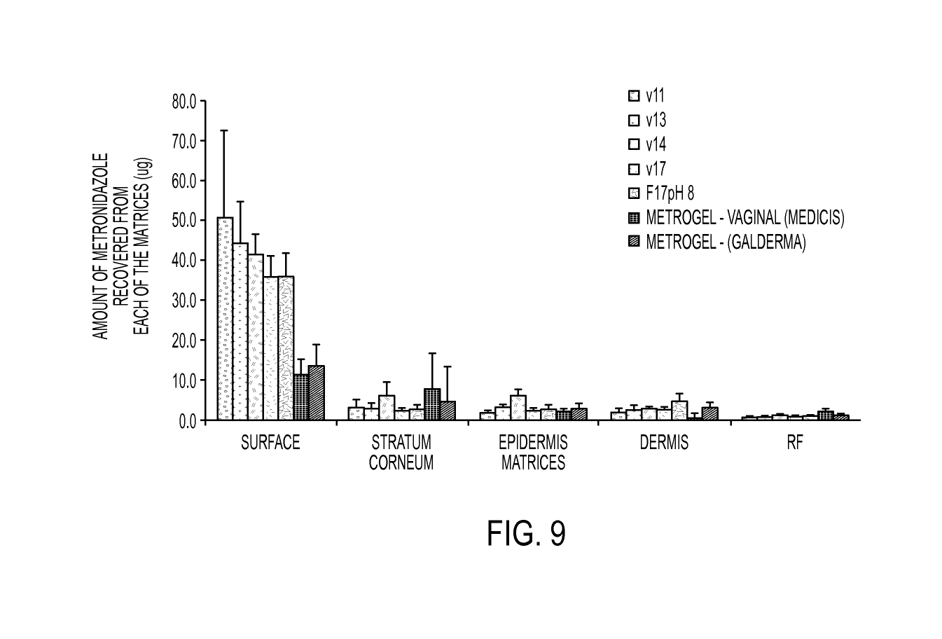

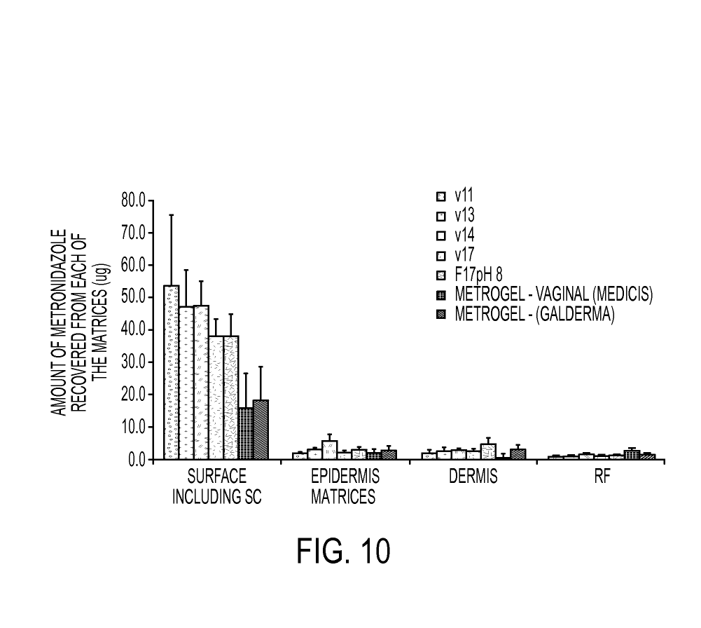

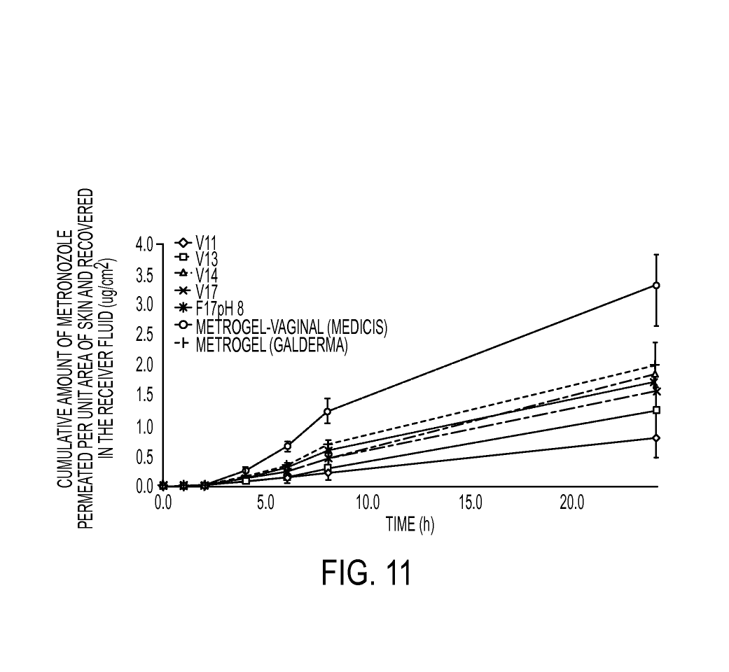

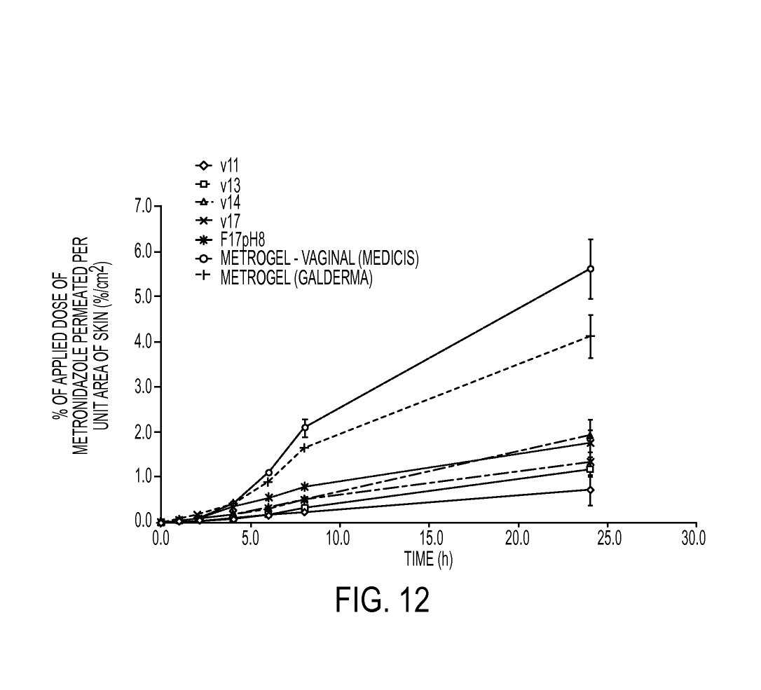

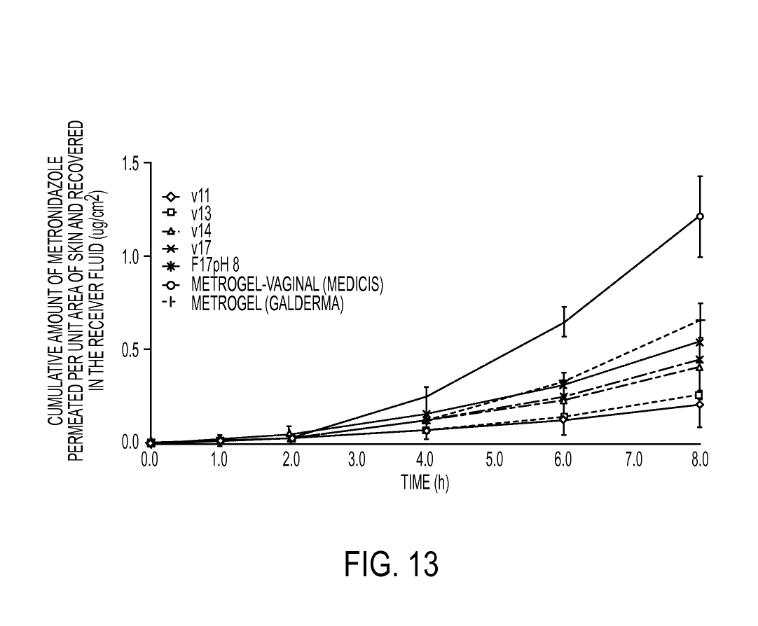

Results are shown in FIGS. 9-13. As shown in FIG. 9, the error bars for the amount of MTZ recovered from the stratum corneum for the control formulations Metrogel and Metrogel-Vaginal are extremely large due to variability in the data. As a consequence, it is not possible to draw any meaningful conclusions regarding the deposition of MTZ in the stratum corneum for any of formulations v11, v13, v14, or v17 versus these controls. However, the data illustrate that all test gels exhibited similar behavior and confirm the general trend of Example 4, i.e. that the formulations described herein selectively and preferentially deliver MTZ to the surface and upper layers of the skin and do not induce penetration into the receiver fluid in Franz Cell tests.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of treating a subject suffering from and/or diagnosed with rosacea, comprising topically applying to the affected area an amount of a high dosage MTZ gel at a frequency and for a total number of applications sufficient to provide therapeutic benefit, where the high dosage MTZ gel comprises about 1.25% to about 2% by weight MTZ, one or more gelling polymers, and water.

2. The method of claim 1, wherein the levels of MTZ from the high dosage MTZ gel measured in the stratum corneum and receiver fluid in an in vitro Franz cell skin permeation experiment carried out with ¹⁴C-labeled MTZ and human cadaver skin are at least about 25 to 55-fold higher and at least about 1 to 20-fold lower, respectively, when normalized for concentration, than the MTZ levels measured from 0.75% METROGEL®.

3-4. (canceled)

5. The method of claim 1, in which the high dosage MTZ gel comprises about 35 wt % to about 40 wt % water.

6. The method of claim 1, in which the high dosage MTZ gel is substantially free of dextrins, cyclodextrins, niacin, and niacinamide, and optionally also surfactants.

7-25. (canceled)

26. The method of claim 1, in which the high dosage MTZ gel has a pH in the range of about pH 5.5 to about pH 6.5.

27. (canceled)

28. The method of claim 1, in which the amount of high dosage MTZ gel applied in a single application contains about 3.75 mg MTZ.

29. The method of claim 1, in which the high dosage MTZ gel includes about 1.5 wt % MTZ.

30. The method of claim 29, in which the high dosage MTZ gel is applied once or twice per day for a period of 4 to 12 weeks.

31. The method of claim 30, in which the high dosage MTZ gel is applied once per day.

32. The method of claim 29, in which the high dosage MTZ gel is applied every other day for a period of 4 to 12, weeks.

33. The method of claim 30, in which the high dosage MTZ gel is applied twice weekly for a period of 4 to 12 weeks.

34. The method of claim 33, in which the high dosage MTZ gel is applied once weekly for a period of 4 to 12 weeks.

35-40. (canceled)

41. The method of claim 29, in which the amount of high dosage MTZ gel applied in a single application contains about 3.75 mg MTZ.

42. A method of treating a subject suffering from and/or diagnosed with rosacea, comprising applying topically to the affected area an amount of a high dosage MTZ gel sufficient to provide therapeutic benefit, wherein the high dosage MTZ gel comprises one or more gelling polymers, about 1.5 wt % MTZ and water, and has one or more features or characteristics selected from the following group:
(a) the levels of MTZ from the high dosage MTZ gel measured in the stratum corneum and receiver fluid in an in vitro Franz cell skin permeation experiment carried out with ¹⁴C-labeled MTZ and human cadaver skin are at least about 25 to 55-fold higher and at least about 1 to 20-fold lower, respectively, when normalized for concentration, than the MTZ levels measured from 0.75% METROGEL®;
(b) a viscosity ranging from about 60,000 mPa to about 500,000 mPa, measured at 25° C. using the controlled shear rate ramp method, a Bohlin CVO 100 rheometer and the rheometer parameters of Table 7;
(c) comprises about 30 wt % to about 60 wt % water;
(d) is substantially free of dextrins, cyclodextrins, niacin, and niacinamide, and optionally also surfactants;
(e) is stable for a period of at least 6 months at 25° C.;
(f) includes about 1 wt % to about 3 wt % total gelling polymers; and
(g) includes about 55% by weight to about 60% by weight of a solvent system for the MTZ.

43. The method of claim 42, in which the high dosage MTZ gel is applied once or twice daily for a period of 4 to 12 weeks.

44. The method of claim 42, where the high dosage MTZ gel is applied for a first course of treatment, treatment discontinued for a period of time, and the treatment resumed for at least a second course of treatment.

45. The method of claim 44, in which the treatment is discontinued for a period of days, weeks, one month, two months, three months, or longer.

46. A high dosage MTZ gel comprising about 1 wt % to about 2 wt % MTZ, about 1 wt % to about 3 wt % of one or more gelling polymers, about 30 wt % to about 60 wt % of a solvent system for the MTZ, and water, wherein:
(a) the levels of MTZ from the high dosage MTZ gel measured in the stratum corneum and receiver fluid in an in vitro Franz cell skin permeation experiment carried out with ¹⁴C-labeled MTZ and human cadaver skin are at least about 25 to 55-fold higher and at least about 1 to 20-fold lower, respectively, when normalized for concentration, than the MTZ levels measured from 0.75% METROGEL®;
(b) wherein the high dosage MTZ gel has a viscosity ranging from about 60,000 mPa to about 500,000 mPa, measured at 25° C. using the controlled shear ramp method, a Bohlin CVO 100 rheometer, and the rheometer parameters of Table 7;
(c) wherein the high dosage MTZ gel comprises about 35 wt % to about 40 wt % water;
(d) wherein the high dosage MTZ gel is substantially free of dextrins, cyclodextrins, niacin and niacinamide, and optionally also surfactants; or
(e) wherein the high dosage MTZ gel is stable for a period of at least 6 months at 25° C.

47-69. (canceled)

70. The gel of claim 46 which has a pH in a range of about pH 5.5 to about pH 6.5.

71. The gel of claim 46 which includes about 1.5% by weight MTZ.

72-74. (canceled)
